SEQUENCE LISTING (48 sequences)

SEQ ID NO: 1 - protein, 263 aa, Artificial Sequence
  GPC3 scFv
MALPVTALLLPLALLLHAARPDVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 2 - protein, 242 aa, Artificial Sequence
  CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 246 aa, Artificial Sequence
  BCMA scFv
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 4 - protein, 259 aa, Artificial Sequence
  MSLN scFv
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 5 - protein, 241 aa, Artificial Sequence
  HER2 scFv
QVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 6 - protein, 46 aa, Artificial Sequence
  CD8 hinge region
TTTPAPRPPTPAPTIASQPLSLRPEACRPAAGGAVHTRGLDFACDI

SEQ ID NO: 7 - protein, 23 aa, Artificial Sequence
  CD8 transmembrane domain
YIWAPLAGTCGVLLLSLVITLYC

SEQ ID NO: 8 - protein, 41 aa, Artificial Sequence
  CD28 intracellular domain
RSKRSRLLHSDYMNMTPRRPGPTRKHYQPYAPPRDFAAYRS

SEQ ID NO: 9 - protein, 42 aa, Artificial Sequence
  41BB intracellular domain
KRGRKKLLYIFKQPFMRPVQTTQEEDGCSCRFPEEEEGGCEL

SEQ ID NO: 10 - protein, 112 aa, Artificial Sequence
  CD3zeta intracellular domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 18 aa, Artificial Sequence
  T2A
EGRGSLLTCGDVEENPGP

SEQ ID NO: 12 - protein, 19 aa, Artificial Sequence
  P2A
ATNFSLLKQAGDVEENPGP

SEQ ID NO: 13 - protein, 167 aa, Artificial Sequence
  secretory leptin
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 14 - protein, 267 aa, Artificial Sequence
  membrane-bound leptin
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCSGGGSGGGGSGGGGSGGGGSGGGSLQTTTPAPRPPTPAPTIASQPLSLRPEACRPAAGGAVHTRGLDFACDIDAGLYVIVPVIISSSILLLGTLLISHQR

SEQ ID NO: 15 - protein, 28 aa, Artificial Sequence
  transmembrane domain of leptin receptor
DAGLYVIVPVIISSSILLLGTLLISHQR

SEQ ID NO: 16 - protein, 21 aa, Artificial Sequence
  signal peptide of leptin receptor
MICQKFCVVLLHWEFIYVITA

SEQ ID NO: 17 - protein, 530 aa, Artificial Sequence
  leptin receptor
IKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDAGLYVIVPVIISSSILLLGTLLISHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 18 - protein, 370 aa, Artificial Sequence
  LRP6
GEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 19 - protein, 243 aa, Artificial Sequence
  TL6
TNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 20 - protein, 362 aa, Artificial Sequence
  LRP5
GEPPTCSPDQFACATGEIDCIPGAWRCDGFPECDDQSDEEGCPVCSAAQFPCARGQCVDLRLRCDGEADCQDRSDEADCDAICLPNQFRCASGQCVLIKQQCDSFPDCIDGSDELMCEITKPPSDDSPAHSSAIGPVIGIILSLFVMGGVYFVCQRVVCQRYAGANGPFPHEYVSGTPHVPLNFIAPGGSQHGPFTGIACGKSMMSSVSLMGGRGGVPLYDRNHVTGASSSSSSSTKATLYPPILNPPPSPATDPSLYNMDMFYSSNIPATARPYRPYIIRGMAPPTTPCSTDVCDSDYSASRWKASKYYLDLNSDSDPYPPPPTPHSQYLSAEDSCPPSPATERSYFHLFPPPPSPCTDSS

SEQ ID NO: 21 - protein, 231 aa, Artificial Sequence
  TL5
SAIGPVIGIILSLFVMGGVYFVCQRVVCQRYAGANGPFPHEYVSGTPHVPLNFIAPGGSQHGPFTGIACGKSMMSSVSLMGGRGGVPLYDRNHVTGASSSSSSSTKATLYPPILNPPPSPATDPSLYNMDMFYSSNIPATARPYRPYIIRGMAPPTTPCSTDVCDSDYSASRWKASKYYLDLNSDSDPYPPPPTPHSQYLSAEDSCPPSPATERSYFHLFPPPPSPCTDSS

SEQ ID NO: 22 - protein, 26 aa, Artificial Sequence
  linker SG3(SG4)3SG3SLQ
SGGGSGGGGSGGGGSGGGGSGGGSLQ

SEQ ID NO: 23 - protein, 486 aa, Artificial Sequence
  CAR1(GPC3-41BB)
MALPVTALLLPLALLLHAARPDVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 469 aa, Artificial Sequence
  CAR2(BCMA-41BB)
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - DNA, 789 bp, Artificial Sequence
  encoding GPC3 scFv
atggctctgcctgtgaccgctctgctgctgcctctggctctgctgctgcacgccgcaagacctgatgtcgtgatgacccagtccccactgtccctgccagtgacaccaggagagcctgcatccatctcttgccggagctcccagtctctggtgcacagcaacggcaatacctacctgcactggtatctgcagaagccaggccagagcccccagctgctgatctacaaggtgtccaaccggttctctggagtgccagaccggttcagcggctccggctctggcaccgatttcacactgaagatcagcagggtggaggcagaggacgtgggcgtgtactattgctcccagaatacccacgtgccccctacatttggccagggcaccaagctggagatcaagggaggaggaggcagcggcggaggaggctccggcggcggcggctctcaggtgcagctggtgcagtccggagcagaggtgaagaagcctggagccagcgtgaaggtgtcctgtaaggcctctggctacaccttcacagattatgagatgcactgggtgcggcaggcacctggacagggactggagtggatgggcgccctggacccaaagaccggcgatacagcctactctcagaagtttaagggcagggtgaccctgacagccgacgagagcacctccacagcctatatggagctgtctagcctgcgcagcgaggataccgccgtgtactattgcacccgcttctacagttacacttattgggggcagggcactctggtcacagtctcttca

SEQ ID NO: 26 - DNA, 726 bp, Artificial Sequence
  encoding CD19 scFv
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 27 - DNA, 738 bp, Artificial Sequence
  encoding BCMA scFv
gacatcgtgctgacccagagccccgcttctttagctgtgtctttaggagagagggccaccatcaattgtcgtgcctccgagtccgtgtccgtgattggcgcccatttaatccactggtaccagcagaaacccggccagccccccaagctgctgatctatttagccagcaatctggagaccggcgtgcccgccagatttagcggaagcggctccggcaccgattttactttaaccatttcctctttacaagctgaggacgccgccatctactactgtttacagagcagaatctttcctcgtaccttcggccaaggtaccaagctggaaatcaagggatccaccagcggctccggaaagcccggttccggagagggcagcacaaagggccaagttcagctcgtgcagagcggctccgagctgaagaagcccggcgcttccgtgaaggtgagctgcaaggccagcggatacaccttcaccgactactccatcaactgggttcgtcaagctcccggtcaaggtctggagtggatgggctggatcaacaccgaaaccagagagcccgcctatgcctacgacttcagaggtcgtttcgtgttctctttagacacaagcgtgagcaccgcctatttacagatcagctctttaaaggccgaggataccgccgtgtactactgcgctcgtgactacagctacgccatggactactggggccaaggtacactggtgaccgtgtccagc

SEQ ID NO: 28 - DNA, 777 bp, Artificial Sequence
  encoding MSLN scFv
caggtgcagctgcaacagagcggccctggcctggtgacaccttcccagaccctcagcctcacctgcgccatctccggcgattccgtgtccagcaatagcgccacctggaactggatcagacagagccctagcagaggcctggagtggctgggcaggacctattataggagcaagtggtacaacgactacgccgtgtccgtcaagtccagaatgagcatcaaccccgacaccagcaagaatcagttcagcctgcagctgaacagcgtgacccccgaggacacagccgtgtattactgcgccaggggcatgatgacctactactacggcatggacgtgtggggccaaggcaccacagtgaccgtgagcagcggcattctgggcagcggaggaggcggatccggcggcggaggcagcggaggaggaggcagccagcctgtgctgacccagtccagctccctgtccgcttcccctggagcctccgcttccctgacctgcaccctgaggtccggcatcaatgtgggcccctacaggatctactggtatcagcagaagcctggcagccccccccagtacctgctgaactacaagagcgacagcgacaagcagcagggcagcggagtgcctagcagattcagcggatccaaggacgcctccgccaatgctggcgtgctgctgatctccggcctgagaagcgaggacgaagccgactactactgcatgatctggcacagcagcgctgccgtgtttggaggcggcacccaactgacagtcctgtcc

SEQ ID NO: 29 - DNA, 723 bp, Artificial Sequence
  encoding HER2 scFv
caggtccagctgcagcagagcggacccgagctcaagaagcccggcgagaccgtgaagatcagctgtaaggccagcggctacccctttaccaactatggaatgaactgggtgaagcaagctcccggccagggcctgaagtggatgggctggatcaacacaagcaccggagaaagcaccttcgctgacgacttcaagggcagatttgacttctccctcgagacctccgccaacaccgcctacctgcagatcaacaatctgaagtccgaggacagcgctacctacttctgcgccagatgggaggtgtaccatggctacgtcccttattggggccaaggcaccaccgtgaccgtgagcagcggaggaggcggttctggaggcggcggcagcggcggcggcggcagcgacattcagctgacccagtcccacaagttcctgagcacctccgtgggcgacagggtgagcattacctgcaaggccagccaggacgtgtacaatgccgtggcctggtaccagcagaagcctggacagagccccaagctgctgatctacagcgcttcctccaggtacaccggcgtcccttccagatttaccggctccggctccggccccgacttcaccttcaccatcagctccgtgcaggccgaagacctggccgtgtacttctgccaacagcacttcaggacccccttcacctttggcagcggaaccaagctggagatcaaa

SEQ ID NO: 30 - DNA, 138 bp, Artificial Sequence
  encoding CD8 hinge region
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatc

SEQ ID NO: 31 - DNA, 69 bp, Artificial Sequence
  encoding CD8 transmembrane domain
tacatctgggcgcccttggccgggacttgtggggtccttctcctgtcactggttatcaccctttactgc

SEQ ID NO: 32 - DNA, 123 bp, Artificial Sequence
  encoding CD28 intracellular domain
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 33 - DNA, 126 bp, Artificial Sequence
  encoding 41BB intracellular domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 34 - DNA, 339 bp, Artificial Sequence
  encoding CD3zeta intracellular domain
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 35 - DNA, 54 bp, Artificial Sequence
  encoding T2A
gagggcagaggaagtcttctaacatgcggtgacgtggaggagaatcccggccct

SEQ ID NO: 36 - DNA, 57 bp, Artificial Sequence
  encoding P2A
gccaccaattttagcctgctgaaacaggccggagacgtggaggaaaaccccggcccc

SEQ ID NO: 37 - DNA, 501 bp, Artificial Sequence
  encoding secretory leptin
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgc

SEQ ID NO: 38 - DNA, 84 bp, Artificial Sequence
  encoding transmembrane domain of leptin receptor
gatgcaggtttatatgtaattgtgccagtaattatttcctcttccatcttattgcttggaacattattaatatcacaccaaaga

SEQ ID NO: 39 - DNA, 63 bp, Artificial Sequence
  encoding signal peptide of leptin receptor
atgatttgtcaaaaattctgtgtggttttgttacattgggaatttatttatgtgataactgcg

SEQ ID NO: 40 - DNA, 1593 bp, Artificial Sequence
  encoding leptin receptor
ataaaagttcctatgagaggacctgaattttggagaataattaatggagatactatgaaaaaggagaaaaatgtcactttactttggaagcccctgatgaaaaatgactcattgtgcagtgttcagagatatgtgataaaccatcatacttcctgcaatggaacatggtcagaagatgtgggaaatcacacgaaattcactttcctgtggacagagcaagcacatactgttacggttctggccatcaattcaattggtgcttctgttgcaaattttaatttaaccttttcatggcctatgagcaaagtaaatatcgtgcagtcactcagtgcttatcctttaaacagcagttgtgtgattgtttcctggatactatcacccagtgattacaagctaatgtattttattattgagtggaaaaatcttaatgaagatggtgaaataaaatggcttagaatctcttcatctgttaagaagtattatatccatgatcattttatccccattgagaagtaccagttcagtctttacccaatatttatggaaggagtgggaaaaccaaagataattaatagtttcactcaagatgatattgaaaaacaccagagtgatgcaggtttatatgtaattgtgccagtaattatttcctcttccatcttattgcttggaacattattaatatcacaccaaagaatgaaaaagctattttgggaagatgttccgaaccccaagaattgttcctgggcacaaggacttaattttcagaagccagaaacgtttgagcatctttttatcaagcatacagcatcagtgacatgtggtcctcttcttttggagcctgaaacaatttcagaagatatcagtgttgatacatcatggaaaaataaagatgagatgatgccaacaactgtggtctctctactttcaacaacagatcttgaaaagggttctgtttgtattagtgaccagttcaacagtgttaacttctctgaggctgagggtactgaggtaacctatgaggacgaaagccagagacaaccctttgttaaatacgccacgctgatcagcaactctaaaccaagtgaaactggtgaagaacaagggcttataaatagttcagtcaccaagtgcttctctagcaaaaattctccgttgaaggattctttctctaatagctcatgggagatagaggcccaggcattttttatattatcagatcagcatcccaacataatttcaccacacctcacattctcagaaggattggatgaacttttgaaattggagggaaatttccctgaagaaaataatgataaaaagtctatctattatttaggggtcacctcaatcaaaaagagagagagtggtgtgcttttgactgacaagtcaagggtatcgtgcccattcccagccccctgtttattcacggacatcagagttctccaggacagttgctcacactttgtagaaaataatatcaacttaggaacttctagtaagaagacttttgcatcttacatgcctcaattccaaacttgttctactcagactcataagatcatggaaaacaagatgtgtgacctaactgtgtaa

SEQ ID NO: 41 - DNA, 1167 bp, Artificial Sequence
  encoding LRP6
atgggggccgtcctgaggagcctcctggcctgcagcttctgtgtgctcctgagagcggaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctga

SEQ ID NO: 42 - DNA, 804 bp, Artificial Sequence
  encoding TL6
atgggggccgtcctgaggagcctcctggcctgcagcttctgtgtgctcctgagagcgccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctga

SEQ ID NO: 43 - DNA, 1089 bp, Artificial Sequence
  encoding LRP5
ggagagccgcccacctgctccccggaccagtttgcatgtgccacaggggagatcgactgtatccccggggcctggcgctgtgacggctttcccgagtgcgatgaccagagcgacgaggagggctgccccgtgtgctccgccgcccagttcccctgcgcgcggggtcagtgtgtggacctgcgcctgcgctgcgacggcgaggcagactgtcaggaccgctcagacgaggcggactgtgacgccatctgcctgcccaaccagttccggtgtgcgagcggccagtgtgtcctcatcaaacagcagtgcgactccttccccgactgtatcgacggctccgacgagctcatgtgtgaaatcaccaagccgccctcagacgacagcccggcccacagcagtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctctacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgcgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctga

SEQ ID NO: 44 - DNA, 696 bp, Artificial Sequence
  encoding TL5
agtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctctacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgcgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctga

SEQ ID NO: 45 - DNA, 78 bp, Artificial Sequence
  encoding linker SG3(SG4)3SG3SLQ
agtggagggggatctgggggcggggggtctggaggaggtgggagtggggggggaggctccggaggcggctccctccaa

SEQ ID NO: 46 - DNA, 578 bp, Artificial Sequence
  IRES
tccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccaca

SEQ ID NO: 47 - protein, 263 aa, Artificial Sequence
  L1H2 scFv
MALPVTALLLPLALLLHAARPDVVMTQSPLSLPVTLGQPASISCRSSQSLVHSNGYTYLHWYQQRPGQSPRLLIYKVSYRGSGVPDRFSGSGSGTDFTLKISRVEAEDVGVYYCGQSGLTPPTFGSGTKLEIKGGGGSGGGGSGGGGSQVQLVQSGAEVKKPGASVKVSCKASGYDFTAYEMHWVRQAPGQGLEWMGALDPKTGQTAYSQKFQGRVTLTADKSISTAYMELSRLRSDDTVVYYCTRFYSYAYWGQGTLVTVSS

SEQ ID NO: 48 - DNA, 789 bp, Artificial Sequence
  encoding L1H2 scFv
atggctctgcctgtgaccgctctgctgctgcctctggctctgctgctgcacgccgcaagacctgacgtggtgatgacccagtcccctctgtctttacccgttacactgggccagcccgcttccatcagctgcagaagcagccagtctttagtgcacagcaacggctacacctatttacactggtaccagcagagacccggccagagccccagactgctgatctacaaggtgtcataccgtggatccggcgtgcccgataggtttagcggcagcggtagcggcaccgatttcactttaaagatctctcgtgtcgaggccgaggatgtgggcgtttactactgcggccagagcggcctcaccccccctaccttcggctccggcaccaagctggagatcaaaggaggcggaggatctggaggaggagggtctggaggcggcgggagccaagtgcagctggtgcagagcggcgccgaggtgaagaaacccggagccagcgtgaaggtgagctgtaaggccagcggctatgacttcaccgcctacgagatgcactgggtgagacaagctcccggtcaaggtctcgagtggatgggcgctttagatcccaagaccgggcagaccgcctactcccagaagttccaaggtcgtgtgactttaaccgccgacaagagcatctccaccgcctacatggagctgtctcgtctgaggagcgacgataccgtggtgtactattgcactcgtttttactcctacgcctactggggccaaggtacactggtgaccgtgagctcc

(19) United States
(12) Patent Application Publication
(10) Pub. No.: US 2022/0389076 A1
(43) Pub. Date: Dec. 8, 2022

(54) MODIFIED IMMUNE CELL AND USE THEREOF

(71) Applicant: Oricell Therapeutics Co., Ltd., Shanghai (CN)

(72) Inventors: Youguo LING, Shanghai (CN); Hao GUO, Shanghai (CN); Siye CHEN, Shanghai (CN); Huijiao LI, Shanghai (CN); Yue YANG, Shanghai (CN); Xiaopei LI, Shanghai (CN); Qi YANG, Shanghai (CN); Huanfeng YANG, Shanghai (CN); Xiaowen HE, Shanghai (CN)

(73) Assignee: Oricell Therapeutics Co., Ltd., Shanghai (CN)

(21) Appl. No.: 17/764,128

(22) PCT Filed: Sep. 25, 2020
(86) PCT No.: PCT/CN2020/117936

§ 371 Date: Mar. 25, 2022

(30) Foreign Application Priority Data
Sep. 26, 2019 (CN) 201910917070.6

(51) Int. Cl. C07K 14/725; C07K 14/575; C07K 14/72; A61P 35/00; A61K 35/17; C07K 14/705

(52) U.S. Cl. CPC C07K 14/7051; C07K 14/575; C07K 14/72; A61P 35/00; A61K 35/17; C07K 14/70578; A61K 2039/5156

(57) ABSTRACT

Provided is a modified immune cell, comprising a chimeric antigen receptor and/or a coding element therefor, or comprising a T cell receptor and/or a coding element therefor. The immune cell further comprises: leptin and/or a functional fragment thereof; and/or, a leptin receptor and/or a functional fragment thereof, and the expression quantity of the leptin receptor and/or the functional fragment thereof in the immune cell is increased compared with that in an immune cell without the corresponding modification. In addition, also provided is another modified immune cell, comprising leptin and/or a functional fragment thereof, and/or, a leptin receptor and/or a functional fragment thereof, and a low-density lipoprotein-receptor-related protein or a fragment thereof.

### FIELD OF THE INVENTION

The present application relates to the field of biomedicine, and specifically to a modified immune cell and a use thereof.

### BACKGROUND OF THE INVENTION

Tumor is a disease that seriously threatens human health. In recent years, with the progress of molecular biology, genomics and proteomics, a series of molecularly targeted drugs have come out one after another for the treatment of tumors. While with the rapid development of immunology, based on the discovery that the recognition specificity of cytotoxic lymphocytes (CTLs) to target cells depends on the T lymphocyte receptor (TCR), further researches have been conducted, that is, the scFv of antibodies against tumor cell-associated antigens is fused with the T lymphocyte receptor to form a chimeric antigen receptor (CAR), which is genetically modified on the surface of T lymphocytes by means of lentiviral infection for the treatment of tumor. Such CAR-T lymphocytes can selectively target T lymphocytes to tumor cells and specifically kill tumor cells in a non-limiting manner with the major histocompatibility complex (MHC). However, adoptive immunotherapy based on immune effector cells has achieved certain effects in some tumors, but the efficacy in most tumors is still unsatisfactory.

### SUMMARY OF THE INVENTION

The present application provides a modified immune cell, which includes a chimeric antigen receptor and/or a coding element therefor, or including a T cell receptor and/or a coding element therefor, and the modified immune cell further includes:

a leptin and/or a functional fragment thereof and/or,

a leptin receptor and/or a functional fragment thereof, and the expression quantity of the leptin receptor and/or the functional fragment thereof in the modified immune cell is increased compared with that in an immune cell without the corresponding modification.

In some embodiments, the modified immune cell includes lymphocytes.

In some embodiments, the modified immune cell includes T cells.

In some embodiments, the leptin and/or the functional fragment thereof is derived from human.

In some embodiments, the leptin receptor and/or the functional fragment thereof is derived from human.

In some embodiments, the leptin is secretory leptin or membrane-bound leptin.

In some embodiments, the leptin and/or the functional fragment thereof includes an amino acid sequence as set forth in any one of SEQ ID NOs: 13-14.

In some embodiments, the functional fragment of the leptin receptor includes a domain of the leptin receptor selected from a group consisting of: an intracellular region, a BOX 1 region, an FN3 region, a C2 region and a transmembrane region.

In some embodiments, the leptin receptor and/or the functional fragment thereof includes an amino acid sequence as set forth in any one of SEQ ID NOs: 15-17.

In some embodiments, the chimeric antigen receptor does not include the leptin and/or the functional fragment thereof.

In some embodiments, the chimeric antigen receptor does not include the leptin receptor and/or the functional fragment thereof.

In some embodiments, the chimeric antigen receptor includes an intracellular domain, the intracellular domain includes a signaling domain and/or a costimulatory domain.

In some embodiments, the signaling domain includes a signaling domain of CD3zeta.

In some embodiments, the costimulatory domain includes a costimulatory domain of 4-1BB.

In some embodiments, the chimeric antigen receptor includes a hinge region.

In some embodiments, the hinge region includes a hinge region of CD8.

In some embodiments, the chimeric antigen receptor includes a transmembrane region.

In some embodiments, the transmembrane region includes a transmembrane region of CD8.

In some embodiments, the chimeric antigen receptor includes a targeting moiety.

In some embodiments, the targeting moiety includes scFv.

In some embodiments, the targeting moiety specifically binds to and/or recognizes a tumor antigen.

In some embodiments, the targeting moiety specifically binds to and/or recognizes a target selected from a group consisting of: CD19, HER2, Mesothelin, GPC3 and BCMA.

In some embodiments, the chimeric antigen receptor includes an amino acid sequence as set forth in any one of SEQ ID NOs: 23-24.

In another aspect, the present application also provides a pharmaceutical composition, which includes the modified immune cell of the present application and optionally a pharmaceutically acceptable carrier.

In another aspect, the present application also provides a use of the modified immune cell of the present application or the pharmaceutical composition of the present application in the preparation of a drug for treating tumor.

In another aspect, the present application also provides a method for promoting immune cell proliferation, which includes steps of:

1) providing a modified immune cell including a chimeric antigen receptor and/or a coding element therefor, or including a T cell receptor and/or a coding element therefor; and

2) allowing the modified immune cell of 1) to express leptin and/or a functional fragment thereof, and/or increasing the expression quantity of a leptin receptor and/or a functional fragment thereof in the modified immune cell of 1).

In another aspect, the present application also provides a method for promoting the production of memory immune cells, which includes steps of:

1) providing a modified immune cell including a chimeric antigen receptor and/or a coding element therefor, or including a T cell receptor and/or a coding element therefor; and

2) allowing the modified immune cell of 1) to express leptin and/or a functional fragment thereof, and/or increasing the expression quantity of a leptin receptor and/or a functional fragment thereof in the modified immune cell of 1).

In another aspect, the present application also provides a method for enhancing the killing ability of an immune cell against tumor, which includes steps of:

1) providing a modified immune cell including a chimeric antigen receptor and/or a coding element therefor, or including a T cell receptor and/or a coding element therefor; and

2) allowing the modified immune cell of 1) to express leptin and/or a functional fragment thereof, and/or increasing the expression quantity of a leptin receptor and/or a functional fragment thereof in the modified immune cell of 1).

In another aspect, the present application also provides a method for inhibiting an immune cell from expressing an immune negative regulatory protein, which includes steps of:

1) providing a modified immune cell including a chimeric antigen receptor and/or a coding element therefor, or including a T cell receptor and/or a coding element therefor; and

2) allowing the modified immune cell of 1) to express leptin and/or a functional fragment thereof, and/or increasing the expression quantity of a leptin receptor and/or a functional fragment thereof in the modified immune cell of 1).

In some embodiments, the step 2) in the method of the present application includes:

introducing a polynucleotide encoding the leptin and/or the functional fragment thereof into the modified immune cell, and/or introducing a polynucleotide encoding the leptin receptor and/or the functional fragment thereof into the modified immune cell.

In some embodiments, the polynucleotide encoding the leptin and/or the functional fragment thereof, and/or the polynucleotide encoding the leptin receptor and/or the functional fragment thereof is contained in a plasmid.

In some embodiments, the plasmid includes a viral vector.

In some embodiments, the modified immune cell includes T cells.

In some embodiments, the increased expression quantity includes that, the expression quantity of the leptin receptor and/or the functional fragment thereof in the modified immune cell is increased compared with that in an immune cell without the corresponding modification.

In some embodiments, the leptin and/or the functional fragment thereof is derived from human.

In some embodiments, the leptin receptor and/or the functional fragment thereof is derived from human.

In some embodiments, the leptin is secretory leptin or membrane-bound leptin.

In some embodiments, the leptin and/or the functional fragment thereof includes an amino acid sequence as set forth in any one of SEQ ID NOs: 13-14.

In some embodiments, the functional fragment of the leptin receptor includes a domain of the leptin receptor selected from a group consisting of: an intracellular region, a BOX 1 region, an FN3 region, a C2 region and a transmembrane region.

In some embodiments, the leptin receptor and/or the functional fragment thereof includes an amino acid sequence as set forth in any one of SEQ ID NOs: 15-17.

In some embodiments, the chimeric antigen receptor does not include the leptin and/or the functional fragment thereof and/or the leptin receptor and/or the functional fragment thereof.

In some embodiments, the chimeric antigen receptor includes an intracellular domain, the intracellular domain includes a signaling domain and/or a costimulatory domain.

In some embodiments, the signaling domain includes a signaling domain of CD3zeta.

In some embodiments, the costimulatory domain includes a costimulatory domain of 4-1BB.

In some embodiments, the chimeric antigen receptor includes a hinge region.

In some embodiments, the hinge region includes a hinge region of CD8.

In some embodiments, the chimeric antigen receptor includes a transmembrane region.

In some embodiments, the transmembrane region includes a transmembrane region of CD8.

In some embodiments, the chimeric antigen receptor includes a targeting moiety.

In some embodiments, the targeting moiety includes scFv.

In some embodiments, the targeting moiety specifically binds to and/or recognizes a tumor antigen.

In some embodiments, the targeting moiety specifically binds to and/or recognizes a target selected from a group consisting of: CD19, HER2, Mesothelin, GPC3 and BCMA.

In some embodiments, the chimeric antigen receptor includes an amino acid sequence as set forth in any one of SEQ ID NOs: 23-24.

In another aspect, the present application also provides a method for inhibiting tumor cell proliferation and/or killing tumor cell, which includes: contacting the modified immune cell of the present application with the tumor cell.

In some embodiments, the method includes an in vitro method or an ex vivo method.

In some embodiments, the tumor includes solid tumor and/or non-solid tumor.

In some embodiments, the tumor includes B lymphocytic leukemia, breast cancer, stomach cancer, pancreatic cancer, multiple myeloma, mesothelioma, lung cancer and/or liver cancer.

In another aspect, the present application provides another modified immune cell, which includes leptin and/or a functional fragment thereof, and/or, a leptin receptor and/or a functional fragment thereof, and a low-density lipoprotein-receptor-related protein or a fragment thereof.

In some embodiments, the modified immune cell includes a T cell.

In some embodiments, the leptin and/or the functional fragment thereof is derived from human.

In some embodiments, the leptin receptor and/or the functional fragment thereof is derived from human.

In some embodiments, the leptin is secretory leptin or membrane-bound leptin.

In some embodiments, the leptin and/or the functional fragment thereof includes an amino acid sequence as set forth in any one of SEQ ID NOs: 13-14.

In some embodiments, the functional fragment of the leptin receptor includes a domain of the leptin receptor selected from a group consisting of: an intracellular region, a BOX 1 region, an FN3 region, a C2 region and a transmembrane region.

In some embodiments, the leptin receptor and/or the functional fragment thereof includes an amino acid sequence as set forth in any one of SEQ ID NOs: 15-17.

In some embodiments, the low-density lipoprotein-receptor-related protein or the fragment thereof is selected from a group consisting of: a low-density lipoprotein-receptor-related protein 6 or a truncated body thereof, and a low-density lipoprotein-receptor-related protein 5 or a truncated body thereof.

In some embodiments, the truncated body of the low-density lipoprotein-receptor-related protein 6 includes an intracellular region of the low-density lipoprotein-receptor-related protein 6; and/or, the truncated body of the low-density lipoprotein-receptor-related protein 5 includes an intracellular region of the low-density lipoprotein-receptor-related protein 5.

In some embodiments, the truncated body of the low-density lipoprotein-receptor-related protein 6 includes a functional domain selected from a group consisting of: a transmembrane region of the low-density lipoprotein-receptor-related protein 6 and an LDLR region of the low-density lipoprotein-receptor-related protein 6; and/or, wherein the truncated body of the low-density lipoprotein-receptor-related protein 5 includes a functional domain selected from a group consisting of: a transmembrane region of the low-density lipoprotein-receptor-related protein 5 and an LDLR region of the low-density lipoprotein-receptor-related protein 5.

In some embodiments, the low-density lipoprotein-receptor-related protein or the fragment thereof includes an amino acid sequence as set forth in any one of SEQ ID NOs: 18-21.

In some embodiments, the modified immune cell further includes a chimeric antigen receptor or a T cell receptor.

In some embodiments, the chimeric antigen receptor does not include the leptin and/or the functional fragment thereof.

In some embodiments, the chimeric antigen receptor does not include the leptin receptor and/or the functional fragment thereof.

In some embodiments, the chimeric antigen receptor does not include the low-density lipoprotein-receptor-related protein or the fragment thereof.

In some embodiments, the chimeric antigen receptor includes an intracellular domain, the intracellular domain includes a signaling domain and/or a costimulatory domain.

In some embodiments, the signaling domain includes a signaling domain of CD3zeta.

In some embodiments, the costimulatory domain includes a costimulatory domain of 4-1BB.

In some embodiments, the chimeric antigen receptor includes a hinge region.

In some embodiments, the hinge region includes a hinge region of CD8.

In some embodiments, the chimeric antigen receptor includes a transmembrane region.

In some embodiments, the transmembrane region includes a transmembrane region of CD8.

In some embodiments, the chimeric antigen receptor includes a targeting moiety.

In some embodiments, the targeting moiety includes scFv.

In some embodiments, the targeting moiety specifically binds to and/or recognizes a tumor antigen.

In some embodiments, the targeting moiety specifically binds to and/or recognizes a target selected from a group consisting of: CD19, HER2, Mesothelin, GPC3 and BCMA.

In some embodiments, the chimeric antigen receptor includes an amino acid sequence as set forth in any one of SEQ ID NOs: 23-24.

In another aspect, the present application also provides a pharmaceutical composition, which includes the modified immune cell of the present application and optionally a pharmaceutically acceptable carrier.

In another aspect, the present application also provides a use of the modified immune cell of the present application or the pharmaceutical composition of the present application in the preparation of a drug for treating tumor.

In another aspect, the present application also provides a method for promoting immune cell proliferation, which includes steps of:

allowing a modified immune cell to express leptin and/or a functional fragment thereof, and/or, increasing the expression quantity of a leptin receptor and/or a functional fragment thereof in the modified immune cell, and allowing the modified immune cell to express a low-density lipoprotein-receptor-related protein or a fragment thereof.

In another aspect, the present application also provides a method for promoting the production of memory immune cells, which includes steps of:

allowing a modified immune cell to express leptin and/or a functional fragment thereof, and/or, increasing the expression quantity of a leptin receptor and/or a functional fragment thereof in the modified immune cell, and allowing the modified immune cell to express a low-density lipoprotein-receptor-related protein or a fragment thereof.

In another aspect, the present application also provides a method for enhancing the killing ability of an immune cell against tumor, which includes steps of:

allowing a modified immune cell to express leptin and/or a functional fragment thereof, and/or, increasing the expression quantity of a leptin receptor and/or a functional fragment thereof in the modified immune cell, and allowing the modified immune cell to express a low-density lipoprotein-receptor-related protein or a fragment thereof.

In another aspect, the present application also provides a method for inhibiting an immune cell from expressing an immune negative regulatory protein, which includes steps of:

allowing a modified immune cell to express leptin and/or a functional fragment thereof, and/or, increasing the expression quantity of a leptin receptor and/or a functional fragment thereof in the modified immune cell, and allowing the modified immune cell to express a low-density lipoprotein-receptor-related protein or a fragment thereof.

In some embodiments, the methods of the present application include a step of: introducing a polynucleotide encoding the leptin and/or the functional fragment thereof into the modified immune cell, and/or introducing a polynucleotide encoding the leptin receptor and/or the functional fragment thereof into the modified immune cell.

In some embodiments, the methods of the present application include a step of: introducing a polynucleotide encoding the low-density lipoprotein-receptor-related protein or the fragment thereof into the modified immune cell.

In some embodiments, the polynucleotide encoding the leptin and/or the functional fragment thereof, and/or the polynucleotide encoding the leptin receptor and/or the functional fragment thereof is contained in a plasmid.

In some embodiments, the polynucleotide encoding the low-density lipoprotein-receptor-related protein or the fragment thereof is contained in a plasmid.

In some embodiments, the plasmid includes a viral vector.

In some embodiments, the modified immune cell includes T cells.

In some embodiments, the leptin and/or the functional fragment thereof is derived from human.

In some embodiments, the leptin receptor and/or the functional fragment thereof is derived from human.

In some embodiments, the leptin is secretory leptin or membrane-bound leptin.

In some embodiments, the leptin and/or the functional fragment thereof includes an amino acid sequence as set forth in any one of SEQ ID NOs: 13-14.

In some embodiments, the functional fragment of the leptin receptor includes a domain of the leptin receptor selected from a group consisting of: an intracellular region, a BOX 1 region, an FN3 region, a C2 region and a transmembrane region.

In some embodiments, the leptin receptor and/or the functional fragment thereof includes an amino acid sequence as set forth in any one of SEQ ID NOs: 15-17.

In some embodiments, the low-density lipoprotein-receptor-related protein or the fragment thereof is selected from a group consisting of: a low-density lipoprotein-receptor-related protein 6 or a truncated body thereof, and a low-density lipoprotein-receptor-related protein 5 or a truncated body thereof.

In some embodiments, the truncated body of the low-density lipoprotein-receptor-related protein 6 includes an intracellular region of the low-density lipoprotein-receptor-related protein 6; and/or, the truncated body of the low-density lipoprotein-receptor-related protein 5 includes an intracellular region of the low-density lipoprotein-receptor-related protein 5.

In some embodiments, the truncated body of the low-density lipoprotein-receptor-related protein 6 includes a functional domain selected from a group consisting of: a transmembrane region of the low-density lipoprotein-receptor-related protein 6 and an LDLR region of the low-density lipoprotein-receptor-related protein 6; and/or, wherein the truncated body of the low-density lipoprotein-receptor-related protein 5 includes a functional domain selected from a group consisting of: a transmembrane region of the low-density lipoprotein-receptor-related protein 5 and an LDLR region of the low-density lipoprotein-receptor-related protein 5.

In some embodiments, the low-density lipoprotein-receptor-related protein or the fragment thereof includes an amino acid sequence as set forth in any one of SEQ ID NOs: 18-21.

In some embodiments, the modified immune cell further includes a chimeric antigen receptor or a T cell receptor.

In some embodiments, the chimeric antigen receptor does not include the leptin and/or the functional fragment thereof, and/or, the leptin receptor and/or the functional fragment thereof.

In some embodiments, the chimeric antigen receptor does not include the low-density lipoprotein-receptor-related protein or the fragment thereof.

In some embodiments, the chimeric antigen receptor includes an intracellular domain, the intracellular domain includes a signaling domain and/or a costimulatory domain.

In some embodiments, the signaling domain includes a signaling domain of CD3zeta.

In some embodiments, the costimulatory domain includes a costimulatory domain of 4-1BB.

In some embodiments, the chimeric antigen receptor includes a hinge region.

In some embodiments, the hinge region includes a hinge region of CD8.

In some embodiments, the chimeric antigen receptor includes a transmembrane region.

In some embodiments, the transmembrane region includes a transmembrane region of CD8.

In some embodiments, the chimeric antigen receptor includes a targeting moiety.

In some embodiments, the targeting moiety includes scFv.

In some embodiments, the targeting moiety specifically binds to and/or recognizes a tumor antigen.

In some embodiments, the targeting moiety specifically binds to and/or recognizes a target selected from a group consisting of: CD19, HER2, Mesothelin, GPC3 and BCMA.

In some embodiments, the chimeric antigen receptor includes an amino acid sequence as set forth in any one of SEQ ID NOs: 23-24.

In another aspect, the present application provides a method for inhibiting tumor cell proliferation and/or killing tumor cell, which includes: contacting the modified immune cell of the present application with the tumor cell.

In some embodiments, the method includes an in vitro method or an ex vivo method.

In some embodiments, the tumor includes solid tumor and/or non-solid tumor.

In some embodiments, the tumor includes B lymphocytic leukemia, breast cancer, stomach cancer, pancreatic cancer, multiple myeloma, mesothelioma, lung cancer and/or liver cancer.

Those skilled in the art can easily perceive other aspects and advantages of the present application from the detailed description below. In the following detailed description, only exemplary embodiments of the present application are shown and described. As those skilled in the art will recognize, the content of the present application enables those skilled in the art to make changes to the disclosed specific embodiments without departing from the spirit and scope of the invention involved in the present application. Correspondingly, the drawings and descriptions in the specification of the present application are merely exemplary, rather than restrictive.

### BRIEF DESCRIPTION OF THE DRAWINGS

FIGS. 1A-1H show the flow cytometry results of memory T cells of GPC3-targeted modified immune cells of the present application (CCR7+CD45RO−).

FIGS. 2A-2H show the flow cytometry results of memory T cells of GPC3-targeted modified immune cells of the present application (CCR7+CD62L+).

FIGS. 3A-3H show the flow cytometry results of memory T cells of BCMA-targeted modified immune cells of the present application (CCR7+CD45RO−).

FIGS. 4A-4H show the flow cytometry results of memory T cells of BCMA-targeted modified immune cells of the present application (CCR7+CD62L+).

FIGS. 5A-5H show the flow cytometry results of memory T cells of GPC3-targeted modified CD8+ immune cells of the present application in CD4 cells (CCR7+CD45RO−).

FIGS. 6A-6H show the flow cytometry results of memory T cells of GPC3-targeted modified CD4+ immune cells of the present application in CD8 cells (CCR7+CD45RO−).

FIGS. 7A-7F show the expression of negative regulatory proteins by the modified immune cells of the present application.

FIG. 8 shows the amplification of GPC3/MSLN/HER2-associated CAR-T.

FIG. 9 shows the flow cytometry results of memory T cells of MSLN-targeted modified immune cells with different structures of the present application.

FIG. 10 shows the flow cytometry results of memory T cells of HER2-targeted modified immune cells with different structures of the present application.

FIG. 11 shows the flow cytometry results of memory T cells of CD19-targeted modified immune cells with different structures of the present application.

FIG. 12 shows the amplification results of immune cells modified with different structures at different target sites of the present application under repeated stimulation.

FIG. 13 shows the results of tumor suppression and recurrence inhibition in mice with the GPC3-targeted modified immune cells of the present application.

FIG. 14 shows the results of body weight changes in mice with the GPC3-targeted modified immune cells of the present application.

FIG. 15 shows the results of cytokine changes in mice with the GPC3-targeted modified immune cells of the present application.

FIG. 16 shows the results of cell changes in mice with the GPC3-targeted modified immune cells of the present application.

FIG. 17 shows the detection results on the contents of modified cells in the spleen (A) and bone marrow (B) of mice after secondary tumor-loading with the GPC3-targeted modified immune cells of the present application.

FIG. 18 shows the results of tumor suppression and recurrence inhibition in mice with the BCMA-targeted modified immune cells of the present application.

FIG. 19 shows the results of cytokine in mice with the BCMA-targeted modified immune cells of the present application.

### DETAILED DESCRIPTION OF THE EMBODIMENTS

The implementation of the present application will be illustrated in the following specific examples, and other advantages and effects of the present application will be easily known by those familiar with this technology from the content disclosed in the specification.

The following is a further description of the present application: In the present invention, unless otherwise specified, the scientific and technical terms used herein have the meanings commonly understood by those skilled in the art. In addition, protein and nucleic acid chemistry, molecular biology, cell and tissue culture, microbiology, immunology related terms and laboratory procedures used herein are all terms and routine procedures widely used in the corresponding fields. At the same time, in order to better understand the present invention, definitions and explanations of related terms are provided below.

In the present application, the term “immune cell” generally refers to cells participating in immune responses, for example, promoting the immune effector responses. In the present application, the immune cell may include lymphocytes. In addition, in the present application, the immune cell may include T cells, B cells, natural killer (NK) cells, mast cells, and phagocytes derived from bone marrow.

In the present application, the term “Chimeric Antigen Receptor” (CAR) generally refers to a fusion protein comprising an extracellular domain capable of binding an antigen and at least one intracellular domain. CAR is the core component of chimeric antigen receptor T cells (CAR-T), which may include a targeting moiety (for example, the moiety that binds tumor-associated antigens (TAAs)), a hinge region, a transmembrane region and an intracellular domain. In the present application, the CAR may be combined with the activated intracellular domain of a T cell receptor based on the antigen specificity of the antibody. Genetically modified T cells expressing CAR can specifically recognize and eliminate malignant cells expressing the target antigen.

In the present application, the term “T cell receptor” (TCR) generally refers to a T cell antigen receptor, which is a molecular structure that T cells specifically recognize and bind to antigen peptide-MHC molecules, and usually exists in the form of a complex with CD3 molecules on the surface of T cells. The TCR of a majority of T cells is composed of alpha and beta peptide chains, and the TCR of a minority of T cells is composed of gamma and delta peptide chains.

In the present application, the term “encoding” generally refers to the inherent property of a specific sequence of nucleotides in a polynucleotide such as a gene, cDNA, or mRNA as a template for synthesizing other polymers and macromolecules in biological processes, where the polymers and macromolecules have defined nucleotide sequences (i.e., rRNA, tRNA and mRNA) or defined amino acid sequences and biological properties derived therefrom. Therefore, if the transcription and translation of mRNA corresponding to a gene produces a protein in a cell or other biological systems, the gene, cDNA or RNA encodes the protein.

In the present application, the term “leptin” generally refers to a class of protein hormones secreted by adipose tissue. Its precursor consists of 167 amino acid residues, and there are 21 amino acid residue signal peptides at the N-terminal. The signal peptides of the precursor are cut off in blood to become 146 amino acids with a molecular weight of 16 KD, thus forming Leptin. Leptin has a wide range of biological effects, the most important of which is to act on the metabolic regulatory center of the hypothalamus, thereby playing the roles of suppressing appetite, reducing energy intake, increasing energy consumption, and inhibiting fat synthesis.

In the present application, the term “leptin receptor” generally refers to a class of transmembrane receptors, which mainly consist of an extracellular region, a transmembrane region and an intracellular region. There are many forms of human leptin receptors, which are divided into two types: long-type (OB-RL) and short-type (OB-RS). The extracellular region and the transmembrane region of both are exactly identical, and the main difference is the length of the intracellular region and the composition of the amino acid sequences. Long-type receptors of leptin are mainly expressed in hypothalamus, including arcuate nucleus, paraventricular nucleus, dorsomedial hypothalamic nucleus and lateral hypothalamic area, while short-type receptors are mostly distributed in adipose tissue, myocardium, lung, ovary and hematopoietic tissue, etc.

In the present application, the term “functional fragment thereof” generally refers to a fragment of a larger polypeptide or polynucleotide which retains the same activity or capability as that of its larger counterpart. The activity level of the functional fragment may be the same as that of its larger counterpart, or less or more. For example, the functional fragment of leptin may be such a polypeptide which is composed of less amino acids than a full-length leptin protein, but may still retain the activity of the full-length leptin.

In the present application, the term “lymphocyte” usually mainly includes natural killer (NK) cells, T cells, or B cells. NK cells are a class of cytotoxic lymphocytes, representing major components of the innate immune system. NK cells reject tumors and virus-infected cells. NK cells function through apoptosis or programmed cell death methods. NK cells are called “natural killer” because they can kill cells without the need of activation. T cells paly a major role in cell-mediated immunity (without involving antibodies). The T cell receptors (TCRs) of T cells differentiate T cells from other types of lymphocytes. The thymus (a specialized organ of the immune system) is the main factor for the maturation of T cells. There are six types of T cells: helper T cells (e.g., CD4+ cells), cytotoxic T cells (also known as TC, cytotoxic T lymphocytes, CTL, T-killing cells, cytolytic T cells, CD8+T cells, or killing T cells), memory T cells ((i) stem memory TSCM cells (similar to naive cells) are CD45RO−, CCR7+, CD45RA+, CD62L+(L-selectin), CD27+, CD28+, and IL-7Rα+, but also display a large amount of CD95, IL-2Rβ, CXCR3, and LFA-1, and exhibit many functional properties characteristic of memory cells); (ii) central memory TCM cells express L-selectin and CCR7, which secret IL-2, but do not produce IFNγ or IL-4; and (iii) effector memory TEM cells do not express L-selectin or CCR7, but produce effector cytokines like IFNγ and IL-4), regulatory T cells (Treg, inhibitory T cells, or CD4+CD25+ regulatory T cells), natural killer T cells (NKTs) and γδT cells. In another aspect, B cells play a major role in humoral immunity (involving antibodies). B cells produce antibodies and antigens, and function as antigen presenting cells (APCs) and transform into memory B cells after activation by antigen interaction.

In the present application, the term “intracellular domain” generally refers to an intracellular domain of any truncated portion sufficient to transduce an activation signal. In the present application, the intracellular domain may include a signaling domain and/or a costimulatory domain.

In the present application, the term “signaling domain” generally refers to a domain capable of transducing signals located inside the cells. In the present application, the signaling domain can transmit signals into cells. For example, the signaling domain may be a signaling domain of the chimeric antigen receptor. For example, in some embodiments, the signaling domain may be selected from a signaling domain of CD3zeta, a signaling domain of CD3delta and a signaling domain of CD3epsilon.

In the present application, the term “costimulatory domain” generally refers to an intracellular domain that can provide immune costimulatory molecules, and the costimulatory molecules are cell surface molecules required for an effective response of lymphocytes to antigens. In the present application, the costimulatory domain may include a costimulatory domain of 4-1BB, a costimulatory domain of CD27 and/or a costimulatory domain of CD28.

In the present application, the term “hinge region” generally refers to a part of a heavy chain molecule linking the CH1 domain to the CH2 domain. In the present application, the hinge region may be a hinge region of CAR. For example, the hinge region may be selected from a hinge region of CD8, a hinge region of IgG1 and a hinge region of IgG4.

In the present application, the term “transmembrane region” generally refers to a domain of a peptide, a polypeptide or a protein capable of spanning the cytoplasmic membrane. In the present application, the transmembrane region may be a transmembrane region of CAR. For example, the transmembrane region may be selected from a group consisting of: a transmembrane region of CD8, a transmembrane region of CD24 and a transmembrane region of CD28.

In the present application, the term “targeting moiety” generally refers to a type of part that acts against certain specific tissues and cells. For example, the targeting moiety can specifically target tumor antigens. In the present application, the targeting moiety may include antibodies or antigen-binding fragments thereof.

In the present application, the term “specifically bind to and/or recognize” generally refers to measurable and reproducible interactions, such as the binding between targets and antibodies, which can determine the presence of the targets in the presence of a heterogeneous population of molecules, including biomolecules. For example, an antibody specifically binding to a target (which may be an epitope) is an antibody that binds to the target with greater affinity, avidity, easier, and/or for a greater duration than it binds to other targets. In one embodiment, the extent to which the antibody binds to an irrelevant target is lower than about 10% of the extent to which the antibody binds to the target, as measured by, for example, radioimmunoassay (RIA). In some embodiments, the antibody specifically binding to the target has a dissociation constant (KD) of <1×10⁻⁶ M, <1×10⁻⁷ M, <1×10⁻⁸ M, <1×10⁻⁹ M or <1×10⁻¹⁰ M. In some embodiments, the antibody specifically binds to the epitope on the protein, and the epitope is conservative among proteins of different species. In another embodiment, specifical binding may include, but does not require exclusive binding.

In the present application, the term “tumor antigen” generally refers to antigen molecules present on tumor cells or normal cells. The tumor antigen may include: embryonic protein, glycoprotein antigen and squamous cell antigen. The tumor antigen may be selected from a group consisting of: an EDB domain of fibronectin, an EDA domain of fibronectin and cell necrotic regions.

In the present application, the term “antigen-binding fragment” generally refers to fragments with antigen-binding activity. In the present application, the antigen-binding fragment may be selected from a group consisting of: Fab, Fab′, F(ab′)₂, F(ab)₂, dAb, an isolated complementarity determining region (CDR), Fv and scFv.

In the present application, the term “pharmaceutical composition” generally refers to a composition suitable for administration to patients, human patients. For example, the pharmaceutical composition of the present application may include the modified immune cell and optionally a pharmaceutically acceptable carrier. In some embodiments, the acceptable ingredients of the pharmaceutical composition are not toxic to recipients at the doses and concentrations used. The pharmaceutical composition of the present application includes, but not limited to, liquid, frozen and lyophilized composition.

In the present application, the term “pharmaceutically acceptable carrier” generally refers to one of the components of the pharmaceutical composition, which may include a buffering agent, an antioxidant, a preservative, a low molecular weight polypeptide, a protein, a hydrophilic polymer, an amino acid, a carbohydrate, a chelating agent, a counter-ion, a metal complex and/or a nonionic surfactant and the like. For example, the pharmaceutically acceptable carrier may include an excipient. For example, the excipient may be selected from a group consisting of: starch, dextrin, sucrose, lactose, magnesium stearate, calcium sulfate, carboxymethyl cellulose, talc, calcium alginate gel, chitosan and nanospheres, and the like. For example, the pharmaceutically acceptable carrier may also be selected from a group consisting of: a pH regulator, an osmotic pressure regulator, a solubilizer and a bacteriostatic agent.

In the present application, the term “tumor” generally refers to neoplasm or solid lesion formed by the growth of abnormal cells. In the present application, the tumor may be solid tumor or blood tumor. For example, the tumor may be selected from a group consisting of: B lymphocytic leukemia, breast cancer, stomach cancer, pancreatic cancer, multiple myeloma, mesothelioma, lung cancer and liver cancer.

In the present application, the term “subject” generally refers to human or non-human animals, including, but not limited to, cat, dog, horse, pig, cow, sheep, rabbit, mouse, rat, or monkey.

In the present application, the term “administration” generally refers to giving the subject (e.g., the patient) a certain dose of substances. Administration can be conducted through any suitable ways, including parenterally, intrapulmonarily and intranasally, as well as (if required by topical treatment) intralesional administration. Parenteral infusion includes, e.g., intramuscular, intravenous, intra-artery, intraperitoneal or subcutaneous administration. Administration may be done through any suitable routes, for example by injection (such as intravenous or subcutaneous injection), depending in part on whether the administration is transient or long-term. Various dosing schedules are contemplated herein, including, but not limited to, a single administration or multiple administrations at various time points, bolus administration, and pulse infusion.

In the present application, the term “low-density lipoprotein-receptor-related protein (LRP)” generally refers to a class of cell surface proteins, belonging to a kind of endocytic receptors, which is one member of low-density-lipoprotein receptor (LDLR) gene family. In the present application, the low-density lipoprotein-receptor-related protein or the fragment there of may be selected from a group consisting of: a low-density lipoprotein-receptor-related protein 6 or a truncated body thereof, and a low-density lipoprotein-receptor-related protein 5 or a truncated body thereof.

In the present application, the term “low-density lipoprotein-receptor-related protein 6” or “LRP6” generally refers to any native LRP6 deriving from any vertebrate sources (including mammal, such as primates (e.g., human) and rodents (e.g., mice and rats)), unless otherwise indicated. This term covers “full-length” unprocessed LRP6 as well as any forms of LRP6 produced by intracellular processing. This term also includes naturally occurring LRP6 variants, e.g., splice variants or allelic variants.

In the present application, the term “low-density lipoprotein-receptor-related protein 5” or “LRP5” generally refers to any native LRP5 deriving from any vertebrate sources (including mammal, such as primates (e.g., human) and rodents (e.g., mice and rats)), unless otherwise indicated. This term covers “full-length” unprocessed LRP5 as well as any forms of LRP5 produced by intracellular processing. This term also includes naturally occurring LRP5 variants, e.g., splice variants or allelic variants.

In the present application, the term “truncated body” generally refers to anything less than the whole. For example, the amino acid sequence of the truncated body of the low-density lipoprotein-receptor-related protein 6 may include any amino acid sequences shorter than the full-length or intact low-density lipoprotein-receptor-related protein 6. For example, in the present application, the truncated body of the low-density lipoprotein-receptor-related protein 6 may include an intracellular region, a transmembrane region or an LDLR region of the low-density lipoprotein-receptor-related protein 6. For another example, the truncated body of the low-density lipoprotein-receptor-related protein 5 may include an intracellular region, a transmembrane region or an LDLR region of the low-density lipoprotein-receptor-related protein 5.

In the present application, the term “plasmid” generally refers to DNA molecules other than chromosomes or nucleoids in organisms such as bacteria and yeast. The plasmids may exist in the cytoplasm and have the ability to replicate autonomously, enabling them to maintain a constant copy number in daughter cells and express the genetic information they carry. The plasmids are used as gene vectors in genetic engineering research. For example, in the present application, the polynucleotide encoding the leptin and/or the functional fragment thereof, and/or the polynucleotide encoding the leptin receptor and/or the functional fragment thereof may be contained in a plasmid. In the present application, the plasmid may include a viral vector.

In the present application, the term “retroviral vector” generally refers to that it can clone and express foreign genes, but cannot self-package into proliferative viral particles. Most of these viruses have reverse transcriptase. Retrovirus contains at least three kinds of genes: gag, genes containing proteins that make up the center and structure of the virus; pol, genes containing reverse transcriptase and env, containing genes that make up the viral coat. Through retroviral transfection, retroviral vectors can randomly and stably integrate their own genome and the foreign genes they carry into the host cell genome, for example, they can integrate CAR molecules into the host cells.

In the present application, the term “lentiviral vector” generally refers to a diploid RNA viral vector that is a retrovirus. Lentiviral vectors are vectors prepared by, based on the genome of the lentivirus, removing multiple sequence structures related to the viral activity to make it biologically safe, and then introducing the sequence and expression structure of the target gene required for the experiment into the genome backbone. Through lentiviral vector transfection, retroviral vectors can randomly and stably integrate their own genome and the foreign genes they carry into the host cell genome, for example, they can integrate CAR molecules into the host cells.

In the present application, the term “transposon” generally refers to a discrete DNA fragment containing a transposase gene. The flank is a terminal inverted repeat (TIR) containing a transposase binding site. The transposase can bind to the TIR and transfer the transposon to a new site.

In the present application, the term “homologue” generally refers to proteins or polypeptides having at least about 80% (e.g., at least about 80%, about 83%, about 85%, about 90%, about 91%, about 92%, about 93%, about 94%, about 95%, about 96%, about 97%, about 98%, about 99% or higher) sequence homology to the amino acid sequence of the protein and/or the polypeptide.

In the present application, the terms “identity” or “homology” are used interchangeably, which generally refers to the similarity, likeness or correlation between two or more sequences. The “percentage of sequence homology” can be calculated by: comparing two sequences to be aligned in a comparison window to determine the number of positions where the same nucleic acid bases (e.g., A, T, C, G, I) or the same amino acid residues (e.g., Ala, Pro, Ser, Thr, Gly, Val, Leu, Ile, Phe, Tyr, Trp, Lys, Arg, His, Asp, Glu, Asn, Gln, Cys and Met) are present in both sequences so as to obtain the number of matching positions, dividing the number of matching positions by the total number of positions in the comparison window (i.e., window size), and multiplying the result by 100, to generate the percentage of sequence homology. The alignment for determining the percentage of sequence homology can be achieved in a variety of ways known in the art, for example, by using publicly available computer softwares, such as BLAST, BLAST-2, ALIGN or Megalign (DNASTAR) softwares. A person skilled in the art can determine appropriate parameters for aligning sequences, including any algorithms needed to achieve the maximum alignment over the full-length sequence range being compared or within the target sequence region. The homology can also be determined by the following methods: PASTA and BLAST. A description of PASTA algorithm can be found in “Improved tools for biological sequence comparison” to W. R. Pearson and D. J. Lipman, Proc. Natl. Acad. Sci., 85: 2444-2448, 1988; and “Rapid and Sensitive Protein Similarity Searches” to D. J. Lipman and W. R. Pearson, Science, 227: 1435-1441, 1989. A description of BLAST algorithm can be found in “Basic Local Alignment Search Tool” to S. Altschul, W. Gish, W. Miller, E. W. Myers and D. Lipman, Journal of Molecular Biology, 215: 403-410, 1990.

In the present application, the term “include” generally refers to the inclusion of explicitly specified features, but not excluding other elements.

In the present application, the term “about” generally refers to varying in a range of 0.5%-10% above or below a specified value, for example, varying in a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below a specified value.

Modified Immune Cell

In one aspect, the present application provides a modified immune cell, which includes a chimeric antigen receptor and/or a coding element therefor, or including a T cell receptor and/or a coding element therefor, and the modified immune cell further includes: leptin and/or a functional fragment thereof; and/or, a leptin receptor and/or a functional fragment thereof, and the expression quantity of the leptin receptor and/or the functional fragment thereof in the modified immune cell is increased compared with that in an immune cell without the corresponding modification.

The expression quantity of the leptin receptor and/or the functional fragment thereof can be determined by methods such as Western Blot (WB), ELISA, flow cytometry, PCR.

In the present application, the modified immune cell may include lymphocytes. In some embodiments, the modified immune cell may include T cells. The T cells may include memory stem cell-like T cells (TSCMs) and/or central memory T cells (TCMs). In some embodiments, the TSCM may be CCR7⁺ and/or CD62L⁺. In some other embodiments, the TSCM may also be selected from a group consisting of: CD45RA⁺ or CD45RA⁻, CD45RO⁺ or CD45RO⁻, CD27⁺, CD28⁺, CD127⁺, CD122⁺, CD95⁺, CD3⁺, CD4⁺ and CD8⁺.

Leptin and Leptin Receptor

In the present application, the leptin and/or the functional fragment thereof may be derived from human. The leptin receptor and/or the functional fragment thereof may also be derived from human.

In the present application, the leptin may be secretory leptin or membrane-bound leptin. For example, the secretory leptin may include an amino acid sequence as set forth in SEQ ID NO: 13. The membrane-bound leptin may include an amino acid sequence as set forth in SEQ ID NO: 14, which may be synthesized by sequentially linking the amino acid sequences as set forth in SEQ ID NO: 13, SEQ ID NO: 22, SEQ ID NO: 6, SEQ ID NO: 15.

In the present application, the leptin and/or the functional fragment thereof may include an amino acid sequence as set forth in any one of SEQ ID NOs: 13-14.

In the present application, the leptin receptor may include sequences with self-activating functions so that the downstream signaling pathway of the leptin receptor can be continuously activated without binding leptin. The functional fragment of the leptin receptor may include a domain of the leptin receptor selected from a group consisting of: an intracellular region, a BOX 1 region, an FN3 region, a C2 region and a transmembrane region. For example, the functional fragment of the leptin receptor may include a leptin receptor transmembrane region (e.g., it may include an amino acid sequence as set forth in SEQ ID NO: 15). For another example, the functional fragment of the leptin receptor may include a leptin receptor signal peptide (e.g., it may include an amino acid sequence as set forth in SEQ ID NO: 16). In addition, the intracellular region of the leptin receptor may be the amino acid sequence at positions 865-1165 in the full-length sequence of the leptin receptor, the BOX 1 region of the leptin receptor may be the amino acid sequence at positions 871-878 in the full-length sequence of the leptin receptor, the FN3 region of the leptin receptor may be the amino acid sequence at positions 732-820 or 636-723 or 536-620 or 235-317 in the full-length sequence of the leptin receptor, the C2 region of the leptin receptor may be the amino acid sequence at positions 329-424 in the full-length sequence of the leptin receptor, the transmembrane region of the leptin receptor may be the amino acid sequence at positions 841-864 in the full-length sequence of the leptin receptor, and the above domains of the leptin receptor have important functions in leptin signaling, protein dimerization or multimer formation.

In the present application, the leptin receptor and/or the functional fragment thereof may include an amino acid sequence as set forth in any one of SEQ ID NOs: 15-17.

In addition, it should be noted that, the sequence of the leptin and/or the functional fragment thereof, and the sequence of the leptin receptor and/or the functional fragment thereof may include one or more amino acid substitutions, additions and/or deletions. For example, the sequence of the leptin and/or the functional fragment thereof may be at least 80%, 85%, 90%, 95%, 96%, 97%, 98% or 99% homologous to its source sequence. For another example, the sequence of the leptin receptor and/or the functional fragment thereof may be at least 80%, 85%, 90%, 95%, 96%, 97%, 98% or 99% homologous to its source sequence.

Chimeric Antigen Receptor

In the present application, the chimeric antigen receptor may not include the leptin and/or the functional fragment thereof. The chimeric antigen receptor may also not include the leptin receptor and/or the functional fragment thereof. In some instances, the chimeric antigen receptor may include the leptin and/or the functional fragment thereof so as to form a protein complex. The chimeric antigen receptor may also include the leptin receptor and/or the functional fragment thereof so as to form a protein complex.

In the present application, the chimeric antigen receptor includes an intracellular domain, and the intracellular domain may include a signaling domain and/or a costimulatory domain. The signaling domain may include one or more selected from a group consisting of: a signaling domain of CD3zeta, a signaling domain of CD3delta and a signaling domain of CD3epsilon. For example, the signaling domain may include a signaling domain of CD3zeta (e.g., it may include an amino acid sequence as set forth in SEQ ID NO: 10). The costimulatory domain includes one or more selected from a group consisting of: a costimulatory domain of 4-1BB, a costimulatory domain of CD27 and a costimulatory domain of CD28. The costimulatory domain may include a costimulatory domain of 4-1BB (e.g., it may include an amino acid sequence as set forth in SEQ ID NO: 9). For another example, the costimulatory domain may include a costimulatory domain of CD28 (e.g., it may include an amino acid sequence as set forth in SEQ ID NO: 8). The nucleotide sequence encoding the costimulatory domain of 4-1BB may be as set forth in SEQ ID NO: 33, and the nucleotide sequence encoding the costimulatory domain of CD28 may be as set forth in SEQ ID NO: 32.

In the present application, the chimeric antigen receptor may include a hinge region. The hinge region may include one or more selected from a group consisting of: a hinge region of CD8, a hinge region of IgG1 and a hinge region of IgG4. For example, the hinge region may include a hinge region of CD8 (e.g., it may include an amino acid sequence as set forth in SEQ ID NO: 6).

In the present application, the chimeric antigen receptor may include a transmembrane region. The transmembrane region may include one or more selected from a group consisting of: a transmembrane region of CD8, a transmembrane region of CD28 and a transmembrane region of CD24. For example, the transmembrane region may include a transmembrane region of CD8 (e.g., it may include an amino acid sequence as set forth in SEQ ID NO: 7).

In the present application, the chimeric antigen receptor may include a targeting moiety. The targeting moiety may include an antibody or an antigen-binding fragment thereof. For example, the targeting moiety may include, but not limited to, recombinant antibody, monoclonal antibody, human antibody, humanized antibody, chimeric antibody, bispecific antibody, single-chain antibody, diabody, triabody, tetrabody, Fv fragment, scFv fragment, Fab fragment, Fab′ fragment, F(ab′)₂ fragment and camelized single-domain antibody.

In the present application, the antibody may include a murine antibody. In some embodiments, the murine antibody or the antigen-binding fragment thereof may also include light chain constant regions of murine kappa, lambda chains or variants thereof, or include heavy chain constant regions of murine IgG1, IgG2, IgG3 or IgG4 or variants thereof.

In the present application, the antibody may be a humanized antibody. In other words, the targeting moiety may be an antibody that immunospecifically binds to an antigen of interest and includes framework regions (FR) substantially having the amino acid sequence of a human antibody and complementarity determining regions (CDRs) substantially having the amino acid sequence of a non-human antibody, or variants, derivatives, analogues or fragments thereof “Substantially” used herein refers to, in the case of CDR, that the amino acid sequence of CDR is at least 80%, at least 85%, at least 90%, at least 95%, at least 98% or at least 99% identical to the amino acid sequence of CDR of a non-human antibody. The humanized antibody may substantially include all, at least one and usually two variable domains (Fab, Fab′, F(ab′)2, FabC, Fv), wherein all or substantially all of the CDR regions correspond to CDR regions of non-human immunoglobulin (i.e., antibodies) and all or substantially all of the framework regions are framework regions having consensus sequences of human immunoglobulin. The humanized antibody also includes at least a part of immunoglobulin constant regions (e.g., Fc), which are generally the constant regions of human immunoglobulin. In some examples, the humanized antibody contains at least one variable domain of a light chain and a heavy chain. The antibody may also include CH1, hinge, CH2, CH3 and CH4 regions of a heavy chain. In some examples, the humanized antibody only contains a humanized light chain. In some examples, the humanized antibody only contains a humanized heavy chain. In a particular example, the humanized antibody only contains a humanized variable domain of a light chain and/or a humanized heavy chain.

In the present application, the antigen-binding fragment may include Fab, Fab′, F(ab)₂, Fv fragments, F(ab′)₂, scFv, di-scFv and/or dAb.

In the present application, the targeting moiety may include scFv. For example, the targeting moiety may include scFv of GPC3, whose amino acid sequence may be as set forth in SEQ ID NO: 1. For example, the targeting moiety may include scFv of L1H2 (targeting GPC3), whose amino acid sequence may be as set forth in SEQ ID NO: 47. For example, the targeting moiety may include scFv of CD19, whose amino acid sequence may be as set forth in SEQ ID NO: 2. For example, the targeting moiety may include scFv of BCMA, whose amino acid sequence may be as set forth in SEQ ID NO: 3. For example, the targeting moiety may include scFv of Mesothelin (or MSLN), whose amino acid sequence may be as set forth in SEQ ID NO: 4. For example, the targeting moiety may include scFv of HER2, whose amino acid sequence may be as set forth in SEQ ID NO: 5. The nucleotide sequence encoding scFv of GPC3 may be as set forth in SEQ ID NO: 25. The nucleotide sequence encoding scFv of CD19 may be as set forth in SEQ ID NO: 26. The nucleotide sequence encoding scFv of BCMA may be as set forth in SEQ ID NO: 27. The nucleotide sequence encoding scFv of Mesothelin (or MSLN) may be as set forth in SEQ ID NO: 28. The nucleotide sequence encoding scFv of HER2 may be as set forth in SEQ ID NO: 29.

In the present application, the targeting moiety may specifically bind to and/or recognize a tumor antigen. In some embodiments, the targeting moiety may specifically bind to and/or recognize a target selected from a group consisting of: CD19, HER2, Mesothelin, GPC3 and BCMA.

In the present application, the chimeric antigen receptor can link splicing proteins. The splicing proteins may be T2A or P2A. For example, the splicing proteins may include an amino acid sequence as set forth in any one of SEQ ID NOs: 11-12.

In the present application, the chimeric antigen receptor can also link ribosome skipping sites. The ribosome skipping sites may be IRES. For example, the ribosome skipping sites may include a nucleotide sequence as set forth in SEQ ID NO: 46.

In the present application, the chimeric antigen receptor may include a targeting moiety, a hinge region, a transmembrane region and an intracellular domain in order from the N-terminus to the C-terminus. For example, the chimeric antigen receptor may include a targeting moiety, a hinge region, a transmembrane region, a costimulatory domain and a signaling domain in order from the N-terminus to the C-terminus. For example, the chimeric antigen receptor may include scFv of GPC3, a hinge region of CD8, a transmembrane region of CD8, a costimulatory domain of 4-1BB and a signaling domain of CD3zeta in order from the N-terminus to the C-terminus. For example, the chimeric antigen receptor may include an amino acid sequence as set forth in SEQ ID NO: 23. For example, the chimeric antigen receptor may be GPC3-41BB. For another example, the chimeric antigen receptor may include scFv of BCMA, a hinge region of CD8, a transmembrane region of CD8, a costimulatory domain of 4-1BB and a signaling domain of CD3zeta in order from the N-terminus to the C-terminus. For example, the chimeric antigen receptor may include an amino acid sequence as set forth in SEQ ID NO: 24. For example, the chimeric antigen receptor may be BCMA-41BB.

In the present application, the chimeric antigen receptor may include an amino acid sequence as set forth in any one of SEQ ID NOs: 23-24.

In addition, it should be noted that, the sequence of the chimeric antigen receptor may include one or more amino acid substitutions, additions and/or deletions. For example, the sequence of the chimeric antigen receptor may be at least 80%, 85%, 90%, 95%, 96%, 97%, 98% or 99% homologous to its source sequence.

Likewise, the sequence of the T cell receptor may include one or more amino acid substitutions, additions and/or deletions. For example, the sequence of the T cell receptor may be at least 80%, 85%, 90%, 95%, 96%, 97%, 98% or 99% homologous to its source sequence.

Modified Immune Cell

In the present application, the modified immune cell may include a chimeric antigen receptor and/or a coding element therefor and leptin and/or a functional fragment thereof. For example, the modified immune cell may be GPC3-41BB-Li, wherein, the amino acid sequence of the chimeric antigen receptor is as set forth in SEQ ID NO: 23, the amino acid sequence of the leptin is as set forth in SEQ ID NO: 13, and the amino acid sequence of the splicing protein is as set forth in SEQ ID NO: 11. For another example, the modified immune cell may be BCMA-41BB-Li, wherein, the amino acid sequence of the chimeric antigen receptor is as set forth in SEQ ID NO: 24, the amino acid sequence of the leptin is as set forth in SEQ ID NO: 13, and the amino acid sequence of the splicing protein is as set forth in SEQ ID NO: 11.

In the present application, the modified immune cell may include a chimeric antigen receptor and/or a coding element therefor and a leptin receptor and/or a functional fragment thereof. For example, the modified immune cell may be GPC3-41BB-LiR, wherein, the amino acid sequence of the chimeric antigen receptor is as set forth in SEQ ID NO: 23, the amino acid sequence of the leptin receptor is as set forth in SEQ ID NO: 17, the amino acid sequence of the leptin receptor signal peptide is as set forth in SEQ ID NO: 16, and the amino acid sequence of the splicing protein is as set forth in SEQ ID NO: 11. For another example, the modified immune cell may be BCMA-41BB-LiR, wherein, the amino acid sequence of the chimeric antigen receptor is as set forth in SEQ ID NO: 24, the amino acid sequence of the leptin receptor is as set forth in SEQ ID NO: 17, the amino acid sequence of the leptin receptor signal peptide is as set forth in SEQ ID NO: 16, and the amino acid sequence of the splicing protein is as set forth in SEQ ID NO: 11.

In the present application, the modified immune cell may include a chimeric antigen receptor and/or a coding element therefor, leptin and/or a functional fragment thereof and a leptin receptor and/or a functional fragment thereof. For example, the modified immune cell may be GPC3-41BB-LiM, wherein, the amino acid sequence of the chimeric antigen receptor is as set forth in SEQ ID NO: 23, the amino acid sequence of the leptin is as set forth in SEQ ID NO: 13, the amino acid sequence of the leptin receptor transmembrane region is as set forth in SEQ ID NO: 15, the amino acid sequence of the splicing protein is as set forth in SEQ ID NO: 11, and the amino acid sequence of the linker is as set forth in SEQ ID NO: 22. For another example, the modified immune cell may be BCMA-41BB-LiM, wherein, the amino acid sequence of the chimeric antigen receptor is as set forth in SEQ ID NO: 24, the amino acid sequence of the leptin is as set forth in SEQ ID NO: 13, the amino acid sequence of the leptin receptor transmembrane region is as set forth in SEQ ID NO: 15, the amino acid sequence of the splicing protein is as set forth in SEQ ID NO: 11, and the amino acid sequence of the linker is as set forth in SEQ ID NO: 22.

In the present application, the modified immune cell may include a T cell receptor and/or a coding element therefor and leptin and/or a functional fragment thereof.

In the present application, the modified immune cell may include a T cell receptor and/or a coding element therefor and a leptin receptor and/or a functional fragment thereof.

In the present application, the modified immune cell may include a T cell receptor and/or a coding element therefor, leptin and/or a functional fragment thereof and a leptin receptor and/or a functional fragment thereof.

In addition, it should be noted that, the sequence of the modified immune cell of the present application may include one or more amino acid substitutions, additions and/or deletions. For example, the sequence of the modified immune cell may be at least 80%, 85%, 90%, 95%, 96%, 97%, 98% or 99% homologous to its source sequence.

Modified Immune Cell

In another aspect, the present application provides another modified immune cell, which includes leptin and/or a functional fragment thereof, and/or, a leptin receptor and/or a functional fragment thereof, and a low-density lipoprotein-receptor-related protein or a fragment thereof.

In the present application, the modified immune cell may include lymphocytes. In some embodiments, the modified immune cell may include T cells. The T cells may include memory stem cell-like T cells (TSCMs) and/or central memory T cells (TCMs). In some embodiments, the TSCMs may be CCR7⁺ and/or CD62L⁺. In some other embodiments, the TSCMs may also be selected from a group consisting of: CD45RA⁺ or CD45RA⁻, CD45RO⁺ or CD45RO⁻, CD27⁺, CD28⁺, CD127⁺, CD122⁺, CD95⁺, CD3⁺, CD4⁺ and CD8⁺.

In the present application, the structures and properties of the leptin and/or the functional fragment thereof and the leptin receptor and/or the functional fragment thereof can be as described above, and will not be repeated here.

In the present application, the low-density lipoprotein-receptor-related protein or the fragment thereof may be selected from a group consisting of: a low-density lipoprotein-receptor-related protein 6 or a truncated body thereof, and a low-density lipoprotein-receptor-related protein 5 or a truncated body thereof. Wherein, the truncated body of the low-density lipoprotein-receptor-related protein 6 may include an intracellular region of the low-density lipoprotein-receptor-related protein 6; and/or, the truncated body of the low-density lipoprotein-receptor-related protein 5 may include an intracellular region of the low-density lipoprotein-receptor-related protein 5. In addition, the truncated body of the low-density lipoprotein-receptor-related protein 6 may also include a functional domain selected from a group consisting of: a transmembrane region of the low-density lipoprotein-receptor-related protein 6 and an LDLR region of the low-density lipoprotein-receptor-related protein 6; and/or, wherein the truncated body of the low-density lipoprotein-receptor-related protein 5 may also include a functional domain selected from a group consisting of: a transmembrane region of the low-density lipoprotein-receptor-related protein 5 and an LDLR region of the low-density lipoprotein-receptor-related protein 5. For example, the low-density lipoprotein-receptor-related protein or the fragment thereof may be a low-density lipoprotein-receptor-related protein 6 (i.e., LRP6), whose amino acid sequence may be as set forth in SEQ ID NO: 18. For example, the low-density lipoprotein-receptor-related protein or the fragment thereof may be the truncated body of a low-density lipoprotein-receptor-related protein 6 (i.e., TL6), whose amino acid sequence may be as set forth in SEQ ID NO: 19. For another example, the low-density lipoprotein-receptor-related protein or the fragment thereof may be a low-density lipoprotein-receptor-related protein 5 (i.e., LRP5), whose amino acid sequence may be as set forth in SEQ ID NO: 20. For another example, the low-density lipoprotein-receptor-related protein or the fragment thereof may be the truncated body of a low-density lipoprotein-receptor-related protein 5 (i.e., TL5), whose amino acid sequence may be as set forth in SEQ ID NO: 21. In addition, the nucleotide sequence encoding LRP6 may be as set forth in SEQ ID NO: 41, the nucleotide sequence encoding TL6 may be as set forth in SEQ ID NO: 42, the nucleotide sequence encoding LRP5 may be as set forth in SEQ ID NO: 43, and the nucleotide sequence encoding TL5 may be as set forth in SEQ ID NO: 44.

In the present application, the low-density lipoprotein-receptor-related protein or the fragment thereof may be a low-density lipoprotein-receptor-related protein 6 or a truncated body thereof. In other instances, the low-density lipoprotein-receptor-related protein or a fragment thereof may be a low-density lipoprotein-receptor-related protein 5 or a truncated body thereof. In some other instances, the low-density lipoprotein-receptor-related protein or the fragment thereof may also be a low-density lipoprotein-receptor-related protein 6 or a truncated body thereof, and a low-density lipoprotein-receptor-related protein 5 or a truncated body thereof.

In the present application, the low-density lipoprotein-receptor-related protein or the fragment thereof may include amino acid sequences as set forth in SEQ ID NOs: 18-21.

In addition, it should be noted that, the sequence of the low-density lipoprotein-receptor-related protein or the fragment thereof may include one or more amino acid substitutions, additions and/or deletions. For example, the sequence of the low-density lipoprotein-receptor-related protein or the fragment thereof may be at least 80%, 85%, 90%, 95%, 96%, 97%, 98% or 99% homologous to its source sequence.

In the present application, the modified immune cell may also include a chimeric antigen receptor or a T cell receptor. The chimeric antigen receptor may not include the leptin and/or the functional fragment thereof. The chimeric antigen receptor may also not include the leptin receptor and/or the functional fragment thereof. In addition, the chimeric antigen receptor may also not include the low-density lipoprotein-receptor-related protein or the fragment thereof. In some instances, the chimeric antigen receptor may include the leptin and/or the functional fragment thereof so as to form a protein complex. The chimeric antigen receptor may also include the leptin receptor and/or the functional fragment thereof so as to form a protein complex. In addition, the chimeric antigen receptor may also include the low-density lipoprotein-receptor-related protein or the fragment thereof so as to form a protein complex. The specific structure and properties of the chimeric antigen receptor can be as described above, and will not be repeated here.

In the present application, the modified immune cell may include a chimeric antigen receptor and/or a coding element therefor and a low-density lipoprotein-receptor-related protein or a fragment thereof. In some embodiments, the modified immune cell may include a chimeric antigen receptor and/or a coding element therefor and a low-density lipoprotein-receptor-related protein 6. For example, the modified immune cell may be GPC3-41BB-L6, wherein, the amino acid sequence of the chimeric antigen receptor is as set forth in SEQ ID NO: 23, the amino acid sequence of the low-density lipoprotein-receptor-related protein 6 is as set forth in SEQ ID NO: 18, and the amino acid sequence of the splicing protein is as set forth in SEQ ID NO: 11. For another example, the modified immune cell may be BCMA-41BB-L6, wherein, the amino acid sequence of the chimeric antigen receptor is as set forth in SEQ ID NO: 24, the amino acid sequence of the low-density lipoprotein-receptor-related protein 6 is as set forth in SEQ ID NO: 18, and the amino acid sequence of the splicing protein is as set forth in SEQ ID NO: 11.

In the present application, the modified immune cell may include a chimeric antigen receptor and/or a coding element therefor, a low-density lipoprotein-receptor-related protein or a fragment thereof and leptin and/or a functional fragment thereof. In some embodiments, the modified immune cell may include a chimeric antigen receptor and/or a coding element therefor, a low-density lipoprotein-receptor-related protein 6 and leptin and/or a functional fragment thereof. For example, the modified immune cell may be GPC3-41BB-L6-Li, wherein, the amino acid sequence of the chimeric antigen receptor is as set forth in SEQ ID NO: 23, the amino acid sequence of the low-density lipoprotein-receptor-related protein 6 is as set forth in SEQ ID NO: 18, the amino acid sequence of the leptin is as set forth in SEQ ID NO: 13, and the amino acid sequence of the splicing protein is as set forth in SEQ ID NO: 11 and SEQ ID NO: 12. For another example, the modified immune cell may be BCMA-41BB-L6-Li, wherein, the amino acid sequence of the chimeric antigen receptor is as set forth in SEQ ID NO: 24, the amino acid sequence of the low-density lipoprotein-receptor-related protein 6 is as set forth in SEQ ID NO: 18, the amino acid sequence of the leptin is as set forth in SEQ ID NO: 13, and the amino acid sequence of the splicing protein is as set forth in SEQ ID NO: 11 and SEQ ID NO: 12.

In the present application, the modified immune cell may include a chimeric antigen receptor and/or a coding element therefor, a low-density lipoprotein-receptor-related protein or a fragment thereof and a leptin receptor and/or a functional fragment thereof. In some embodiments, the modified immune cell may include a chimeric antigen receptor and/or a coding element therefor, a low-density lipoprotein-receptor-related protein 6 and a leptin receptor and/or a functional fragment thereof. For example, the modified immune cell may be GPC3-41BB-L6-LiR, wherein, the amino acid sequence of the chimeric antigen receptor is as set forth in SEQ ID NO: 23, the amino acid sequence of the low-density lipoprotein-receptor-related protein 6 is as set forth in SEQ ID NO: 18, the amino acid sequence of the leptin receptor signal peptide is as set forth in SEQ ID NO: 16, the amino acid sequence of the leptin receptor is as set forth in SEQ ID NO: 17, and the amino acid sequence of the splicing protein is as set forth in SEQ ID NO: 11 and SEQ ID NO: 12. For another example, the modified immune cell may be BCMA-41BB-L6-LiR, wherein, the amino acid sequence of the chimeric antigen receptor is as set forth in SEQ ID NO: 24, the amino acid sequence of the low-density lipoprotein-receptor-related protein 6 is as set forth in SEQ ID NO: 18, the amino acid sequence of the leptin receptor signal peptide is as set forth in SEQ ID NO: 16, the amino acid sequence of the leptin receptor is as set forth in SEQ ID NO: 17, and the amino acid sequence of the splicing protein is as set forth in SEQ ID NO: 11 and SEQ ID NO: 12.

In the present application, the modified immune cell may include a chimeric antigen receptor and/or a coding element therefor, a low-density lipoprotein-receptor-related protein or a fragment thereof, leptin and/or a functional fragment thereof and a leptin receptor and/or a functional fragment thereof. In some embodiments, the modified immune cell may include a chimeric antigen receptor and/or a coding element therefor, a low-density lipoprotein-receptor-related protein 6, leptin and/or a functional fragment thereof and a leptin receptor and/or a functional fragment thereof. For example, the modified immune cell may be GPC3-41BB-L6-LiM, wherein, the amino acid sequence of the chimeric antigen receptor is as set forth in SEQ ID NO: 23, the amino acid sequence of the low-density lipoprotein-receptor-related protein 6 is as set forth in SEQ ID NO: 18, the amino acid sequence of the leptin is as set forth in SEQ ID NO: 13, the amino acid sequence of the leptin receptor transmembrane region is as set forth in SEQ ID NO: 15, the amino acid sequence of the splicing protein is as set forth in SEQ ID NO: 11 and SEQ ID NO: 12, and the amino acid sequence of the linker is as set forth in SEQ ID NO: 22. For another example, the modified immune cell may be BCMA-41BB-L6-LiM, wherein, the amino acid sequence of the chimeric antigen receptor is as set forth in SEQ ID NO: 24, the amino acid sequence of the low-density lipoprotein-receptor-related protein 6 is as set forth in SEQ ID NO: 18, the amino acid sequence of the leptin is as set forth in SEQ ID NO: 13, the amino acid sequence of the leptin receptor transmembrane region is as set forth in SEQ ID NO: 15, the amino acid sequence of the splicing protein is as set forth in SEQ ID NO: 11 and SEQ ID NO: 12, and the amino acid sequence of the linker is as set forth in SEQ ID NO: 22.

In the present application, the modified immune cell may include a T cell receptor and/or a coding element therefor and a low-density lipoprotein-receptor-related protein or a fragment thereof.

In the present application, the modified immune cell may include a T cell receptor and/or a coding element therefor, a low-density lipoprotein-receptor-related protein or a fragment thereof and leptin and/or a functional fragment thereof.

In the present application, the modified immune cell may include a T cell receptor and/or a coding element therefor, a low-density lipoprotein-receptor-related protein or a fragment thereof and a leptin receptor and/or a functional fragment thereof.

In the present application, the modified immune cell may include a T cell receptor and/or a coding element therefor, a low-density lipoprotein-receptor-related protein or a fragment thereof, leptin and/or a functional fragment thereof and a leptin receptor and/or a functional fragment thereof.

In addition, it should be noted that, the sequence of the modified immune cell of the present application may include one or more amino acid substitutions, additions and/or deletions. For example, the sequence of the modified immune cell may be at least 80%, 85%, 90%, 95%, 96%, 97%, 98% or 99% homologous to its source sequence.

Pharmaceutical Composition

In another aspect, the present application also provides a pharmaceutical composition, which may include the modified immune cell of the present application and optionally a pharmaceutically acceptable carrier. Where, the pharmaceutically acceptable carrier may include a buffering agent, an antioxidant, a preservative, a low molecular weight polypeptide, a protein, a hydrophilic polymer, an amino acid, a carbohydrate, a chelating agent, a counter-ion, a metal complex and/or a nonionic surfactant and the like. For example, the pharmaceutically acceptable carrier may include an excipient, for example, the excipient may be selected from a group consisting of: starch, dextrin, sucrose, lactose, magnesium stearate, calcium sulfate, carboxymethyl cellulose, talc, calcium alginate gel, chitosan and nanospheres, and the like. For example, the pharmaceutically acceptable carrier may also be selected from a group consisting of: a pH regulator, an osmotic pressure regulator, a solubilizer and a bacteriostatic agent.

Application and Use

In another aspect, the present application also provides a use of the modified immune cell or the pharmaceutical composition in the preparation of a drug for treating tumor.

In another aspect, the present application provides a method for promoting immune cell proliferation, which includes steps of:

1) providing a modified immune cell including a chimeric antigen receptor and/or a coding element therefor, or including a T cell receptor and/or a coding element therefor; and

2) allowing the modified immune cell of 1) to express leptin and/or a functional fragment thereof, and/or increasing the expression quantity of a leptin receptor and/or a functional fragment thereof in the modified immune cell of 1).

In another aspect, the present application provides a method for promoting the production of memory immune cells, which includes steps of:

1) providing a modified immune cell including a chimeric antigen receptor and/or a coding element therefor, or including a T cell receptor and/or a coding element therefor; and

2) allowing the modified immune cell of 1) to express leptin and/or a functional fragment thereof, and/or increasing the expression quantity of a leptin receptor and/or a functional fragment thereof in the modified immune cell of 1).

In another aspect, the present application provides a method for enhancing the killing ability of an immune cell against tumor, which includes steps of:

1) providing a modified immune cell including a chimeric antigen receptor and/or a coding element therefor, or including a T cell receptor and/or a coding element therefor; and

2) allowing the modified immune cell of 1) to express leptin and/or a functional fragment thereof, and/or increasing the expression quantity of a leptin receptor and/or a functional fragment thereof in the modified immune cell of 1).

In another aspect, the present application provides a method for inhibiting an immune cell from expressing an immune negative regulatory protein, which includes steps of:

1) providing a modified immune cell including a chimeric antigen receptor and/or a coding element therefor, or including a T cell receptor and/or a coding element therefor; and

2) allowing the modified immune cell of 1) to express leptin and/or a functional fragment thereof, and/or increasing the expression quantity of a leptin receptor and/or a functional fragment thereof in the modified immune cell of 1).

In the present application, the immune negative regulatory protein may be TIM3, LAG3, PD1 or PDL1.

In the present application, the increased expression quantity may include that, the expression quantity of the leptin receptor and/or the functional fragment thereof in the modified immune cell is increased compared with that in an immune cell without the corresponding modification. The expression quantity of the leptin receptor and/or the functional fragment thereof can be determined by methods such as Western Blot (WB), ELISA, flow cytometry, PCR.

In the present application, the step 2) may include:

introducing a polynucleotide encoding the leptin and/or the functional fragment thereof into the modified immune cell, and/or introducing a polynucleotide encoding the leptin receptor and/or the functional fragment thereof into the modified immune cell.

In the present application, the polynucleotide encoding the leptin and/or the functional fragment thereof, and/or the polynucleotide encoding the leptin receptor and/or the functional fragment thereof may be contained in a plasmid. The plasmid may include a viral vector. The plasmid may also include a retroviral vector, a lentiviral vector or a transposon plasmid.

In the present application, the modified immune cell may include lymphocytes. In some embodiments, the modified immune cell may include T cells. The T cells may include memory stem cell-like T cells (TSCMs) and/or central memory T cells (TCMs). In some embodiments, the TSCMs may be CCR7⁺ and/or CD62L⁺. In some other embodiments, the TSCMs may also be selected from a group consisting of: CD45RA⁺ or CD45RA⁻, CD45RO⁺ or CD45RO⁻, CD27⁺, CD28⁺, CD127⁺, CD122⁺, CD95⁺, CD3⁺, CD4⁺ and CD8⁺.

In the present application, the structures and properties of the leptin and/or the functional fragment thereof and the leptin receptor and/or the functional fragment thereof can be as described above, and will not be repeated here.

In another aspect, the present application also provides a method for promoting immune cell proliferation, which includes steps of:

allowing a modified immune cell to express leptin and/or a functional fragment thereof, and/or, increasing the expression quantity of a leptin receptor and/or a functional fragment thereof in the modified immune cell, and allowing the modified immune cell to express a low-density lipoprotein-receptor-related protein or a fragment thereof.

In another aspect, the present application also provides a method for promoting the production of memory immune cells, which includes steps of:

allowing a modified immune cell to express leptin and/or a functional fragment thereof, and/or, increasing the expression quantity of a leptin receptor and/or a functional fragment thereof in the modified immune cell, and allowing the modified immune cell to express a low-density lipoprotein-receptor-related protein or a fragment thereof.

In another aspect, the present application also provides a method for enhancing the killing ability of an immune cell against tumor, which includes steps of:

allowing a modified immune cell to express leptin and/or a functional fragment thereof, and/or, increasing the expression quantity of a leptin receptor and/or a functional fragment thereof in the modified immune cell, and allowing the modified immune cell to express a low-density lipoprotein-receptor-related protein or a fragment thereof.

In another aspect, the present application also provides a method for inhibiting an immune cell from expressing an immune negative regulatory protein, which includes steps of:

allowing a modified immune cell to express leptin and/or a functional fragment thereof, and/or, increasing the expression quantity of a leptin receptor and/or a functional fragment thereof in the modified immune cell, and allowing the modified immune cell to express a low-density lipoprotein-receptor-related protein or a fragment thereof.

In the present application, the immune negative regulatory protein may be TIM3, LAG3, PD1 or PDL1.

In the present application, the increased expression quantity may include that, the expression quantity of the leptin receptor and/or the functional fragment thereof in the modified immune cell is increased compared with that in an immune cell without the corresponding modification. The expression quantity of the leptin receptor and/or the functional fragment thereof can be determined by methods such as Western Blot (WB), ELISA, flow cytometry, PCR.

In the present application, the method, including the method for promoting immune cell proliferation, the method for promoting the production of memory immune cells, the method for enhancing the killing ability of an immune cell against tumor or the method for inhibiting an immune cell from expressing an immune negative regulatory protein, may also include steps of: introducing a polynucleotide encoding the leptin and/or the functional fragment thereof into the modified immune cell, and/or introducing a polynucleotide encoding the leptin receptor and/or the functional fragment thereof into the modified immune cell.

In the present application, the method, including the method for promoting immune cell proliferation, the method for promoting the production of memory immune cells, the method for enhancing the killing ability of an immune cell against tumor or the method for inhibiting an immune cell from expressing an immune negative regulatory protein, may also include a step of: introducing a polynucleotide encoding the low-density lipoprotein-receptor-related protein or the fragment thereof into the modified immune cell.

In the present application, the polynucleotide encoding the leptin and/or the functional fragment thereof, and/or the polynucleotide encoding the leptin receptor and/or the functional fragment thereof may be contained in a plasmid.

In the present application, the polynucleotide encoding the low-density lipoprotein-receptor-related protein or the fragment thereof is contained in a plasmid.

The plasmid may include a viral vector. The plasmid may also include a retroviral vector, a lentiviral vector or a transposonplasmid.

In the present application, the modified immune cell may include lymphocytes. In some embodiments, the modified immune cell may include T cells. The T cells may include memory stem cell-like T cells (TSCMs) and/or central memory T cells (TCMs). In some embodiments, the TSCMs may be CCR7⁺ and/or CD62L⁺. In some other embodiments, the TSCMs may also be selected from a group consisting of: CD45RA⁺ or CD45RA⁻, CD45RO⁺ or CD45RO⁻, CD27⁺, CD28⁺, CD127⁺, CD122⁺, CD95⁺, CD3⁺, CD4⁺ and CD8⁺.

In the present application, the structures and properties of the leptin and/or the functional fragment thereof, the leptin receptor and/or the functional fragment thereof and the low-density lipoprotein-receptor-related protein or the fragment thereof can be as described above, and will not be repeated here.

In another aspect, the present application also provides a method for inhibiting tumor cell proliferation and/or killing tumor cell, which includes: contacting the modified immune cell of the present application with the tumor cell. In some embodiments, the contact may be administration, that is, giving the subject (e.g., a patient) a certain dose of the modified immune cell of the present application. Administration can be conducted through any suitable ways, including parenterally, intrapulmonarily and intranasally, as well as (if required by topical treatment) intralesional administration. Parenteral infusion includes, e.g., intramuscular, intravenous, intra-artery, intraperitoneal or subcutaneous administration. Administration may be done through any suitable routes, for example by injection (such as intravenous or subcutaneous injection), depending in part on whether the administration is transient or long-term. Various dosing schedules are contemplated herein, including, but not limited to, a single administration or multiple administrations at various time points, bolus administration, and pulse infusion.

In the present application, the method for inhibiting tumor cell proliferation and/or killing tumor cell, wherein the method may include an in vitro method or an ex vivo method.

In the present application, the tumor includes solid tumor and/or non-solid tumor. For example, the tumor may include B lymphocytic leukemia, breast cancer, stomach cancer, pancreatic cancer, multiple myeloma, mesothelioma, lung cancer and/or liver cancer.

Without intending to be limited by any theory, the following examples are only intended to illustrate the protein molecules, preparation methods and uses of the present application, and are not intended to limit the scope of the present invention. The examples do not include detailed descriptions of conventional methods, such as methods for constructing vectors and plasmids, methods for inserting protein-encoding genes into such vectors and plasmids, or methods for introducing plasmids into host cells.

### EXAMPLES

### Example 1. Construction of Lentiviral Vectors

The CAR-T, with targeting GPC3 and BCMA as examples, artificially synthesizes a fragment containing the CAR structure, and constructs it into a lentiviral vector (LV100A, System Biosciences Co.), and then transfects it according to the method documented in its instruction to obtain a lentivirus, thus obtaining GPC3-41BB, GPC3-41BB-L6, GPC3-41BB-Li, GPC3-41BB-LiM, GPC3-41BB-LiR, GPC3-41BB-L6-Li, GPC3-41BB-L6-LiM, GPC3-41BB-L6-LiR; BCMA-41BB, BCMA-41BB-L6, BCMA-41BB-Li, BCMA-41BB-LiM, BCMA-41BB-LiR, BCMA-41BB-L6-Li, BCMA-41BB-L6-LiM, BCMA-41BB-L6-LiR lentiviruses, respectively.

The amino acid sequence of GPC3-41BB (from the N-terminus to the C-terminus) is assembled by splicing SEQ ID NO: 1, SEQ ID NO: 6, SEQ ID NO: 7, SEQ ID NO: 9, SEQ ID NO: 10 in order.

The amino acid sequence of GPC3-41BB-L6 (from the N-terminus to the C-terminus) is assembled by splicing SEQ ID NO: 1, SEQ ID NO: 6, SEQ ID NO: 7, SEQ ID NO: 9, SEQ ID NO: 10, SEQ ID NO: 11, SEQ ID NO: 18 in order.

The amino acid sequence of GPC3-41BB-Li (from the N-terminus to the C-terminus) is assembled by splicing SEQ ID NO: 1, SEQ ID NO: 6, SEQ ID NO: 7, SEQ ID NO: 9, SEQ ID NO: 10, SEQ ID NO: 11, SEQ ID NO: 13 in order.

The amino acid sequence of GPC3-41BB-LiM (from the N-terminus to the C-terminus) is assembled by splicing SEQ ID NO: 1, SEQ ID NO: 6, SEQ ID NO: 7, SEQ ID NO: 9, SEQ ID NO: 10, SEQ ID NO: 11, SEQ ID NO: 13, SEQ ID NO: 22, SEQ ID NO: 6, SEQ ID NO: 15 in order.

The amino acid sequence of GPC3-41BB-LiR (from the N-terminus to the C-terminus) is assembled by splicing SEQ ID NO: 1, SEQ ID NO: 6, SEQ ID NO: 7, SEQ ID NO: 9, SEQ ID NO: 10, SEQ ID NO: 11, SEQ ID NO: 16, SEQ ID NO: 17 in order.

The amino acid sequence of GPC3-41BB-L6-Li (from the N-terminus to the C-terminus) is assembled by splicing SEQ ID NO: 1, SEQ ID NO: 6, SEQ ID NO: 7, SEQ ID NO: 9, SEQ ID NO: 10, SEQ ID NO: 11, SEQ ID NO: 18, SEQ ID NO: 12, SEQ ID NO: 13 in order.

The amino acid sequence of GPC3-41BB-L6-LiM (from the N-terminus to the C-terminus) is assembled by splicing SEQ ID NO: 1, SEQ ID NO: 6, SEQ ID NO: 7, SEQ ID NO: 9, SEQ ID NO: 10, SEQ ID NO: 11, SEQ ID NO: 18, SEQ ID NO: 12, SEQ ID NO: 13, SEQ ID NO: 22, SEQ ID NO: 6, SEQ ID NO: 15 in order.

The amino acid sequence of GPC3-41BB-L6-LiR (from the N-terminus to the C-terminus) is assembled by splicing SEQ ID NO: 1, SEQ ID NO: 6, SEQ ID NO: 7, SEQ ID NO: 9, SEQ ID NO: 10, SEQ ID NO: 11, SEQ ID NO: 18, SEQ ID NO: 12, SEQ ID NO: 16, SEQ ID NO: 17 in order.

The amino acid sequence of L1H2-41BB-L6 (from the N-terminus to the C-terminus) is assembled by splicing SEQ ID NO: 47, SEQ ID NO: 6, SEQ ID NO: 7, SEQ ID NO: 9, SEQ ID NO: 10, SEQ ID NO: 11, SEQ ID NO: 18 in order.

The amino acid sequence of L1H2-41BB-LiR (from the N-terminus to the C-terminus) is assembled by splicing SEQ ID NO: 47, SEQ ID NO: 6, SEQ ID NO: 7, SEQ ID NO: 9, SEQ ID NO: 10, SEQ ID NO: 11, SEQ ID NO: 16, SEQ ID NO: 17 in order.

The amino acid sequence of L1H2-41BB-L6-Li (from the N-terminus to the C-terminus) is assembled by splicing SEQ ID NO: 47, SEQ ID NO: 6, SEQ ID NO: 7, SEQ ID NO: 9, SEQ ID NO: 10, SEQ ID NO: 11, SEQ ID NO: 18, SEQ ID NO: 12, SEQ ID NO: 13 in order.

The amino acid sequence of L1H2-41BB-L6-LiM (from the N-terminus to the C-terminus) is assembled by splicing SEQ ID NO: 47, SEQ ID NO: 6, SEQ ID NO: 7, SEQ ID NO: 9, SEQ ID NO: 10, SEQ ID NO: 11, SEQ ID NO: 18, SEQ ID NO: 12, SEQ ID NO: 13, SEQ ID NO: 22, SEQ ID NO: 6, SEQ ID NO: 15 in order. Correspondingly, the nucleotide sequence of GPC3-41BB (from the N-terminus to the C-terminus) is assembled by splicing SEQ ID NO: 25, SEQ ID NO: 30, SEQ ID NO: 31, SEQ ID NO: 33, SEQ ID NO: 34 in order.

The nucleotide sequence of GPC3-41BB-L6 (from the N-terminus to the C-terminus) is assembled by splicing SEQ ID NO: 25, SEQ ID NO: 30, SEQ ID NO: 31, SEQ ID NO: 33, SEQ ID NO: 34, SEQ ID NO: 35, SEQ ID NO: 41 in order.

The nucleotide sequence of GPC3-41BB-Li (from the N-terminus to the C-terminus) is assembled by splicing SEQ ID NO: 25, SEQ ID NO: 30, SEQ ID NO: 31, SEQ ID NO: 33, SEQ ID NO: 34, SEQ ID NO: 35, SEQ ID NO: 37 in order.

The nucleotide sequence of GPC3-41BB-LiM (from the N-terminus to the C-terminus) is assembled by splicing SEQ ID NO: 25, SEQ ID NO: 30, SEQ ID NO: 31, SEQ ID NO: 33, SEQ ID NO: 34, SEQ ID NO: 35, SEQ ID NO: 37, SEQ ID NO: 45, SEQ ID NO: 30, SEQ ID NO: 38 in order.

The nucleotide sequence of GPC3-41BB-LiR (from the N-terminus to the C-terminus) is assembled by splicing SEQ ID NO: 25, SEQ ID NO: 30, SEQ ID NO: 31, SEQ ID NO: 33, SEQ ID NO: 34, SEQ ID NO: 35, SEQ ID NO: 39, SEQ ID NO: 40 in order.

The nucleotide sequence of GPC3-41BB-L6-Li (from the N-terminus to the C-terminus) is assembled by splicing SEQ ID NO: 25, SEQ ID NO: 30, SEQ ID NO: 31, SEQ ID NO: 33, SEQ ID NO: 34, SEQ ID NO: 35, SEQ ID NO: 41, SEQ ID NO: 36, SEQ ID NO: 37 in order.

The nucleotide sequence of GPC3-41BB-L6-LiM (from the N-terminus to the C-terminus) is assembled by splicing SEQ ID NO: 25, SEQ ID NO: 30, SEQ ID NO: 31, SEQ ID NO: 33, SEQ ID NO: 34, SEQ ID NO: 35, SEQ ID NO: 41, SEQ ID NO: 36, SEQ ID NO: 37, SEQ ID NO: 45, SEQ ID NO: 30, SEQ ID NO: 38 in order.

The nucleotide sequence of GPC3-41BB-L6-LiR (from the N-terminus to the C-terminus) is assembled by splicing SEQ ID NO: 25, SEQ ID NO: 30, SEQ ID NO: 31, SEQ ID NO: 33, SEQ ID NO: 34, SEQ ID NO: 35, SEQ ID NO: 41, SEQ ID NO: 36, SEQ ID NO: 39, SEQ ID NO: 40 in order.

The nucleotide sequence of L1H2-41BB-L6 (from the N-terminus to the C-terminus) is assembled by splicing SEQ ID NO: 48, SEQ ID NO: 30, SEQ ID NO: 31, SEQ ID NO: 33, SEQ ID NO: 34, SEQ ID NO: 35, SEQ ID NO: 41 in order.

The nucleotide sequence of L1H2-41BB-LiR (from the N-terminus to the C-terminus) is assembled by splicing SEQ ID NO: 48, SEQ ID NO: 30, SEQ ID NO: 31, SEQ ID NO: 33, SEQ ID NO: 34, SEQ ID NO: 35, SEQ ID NO: 39, SEQ ID NO: 40 in order.

The nucleotide sequence of L1H2-41BB-L6-Li (from the N-terminus to the C-terminus) is assembled by splicing SEQ ID NO: 48, SEQ ID NO: 30, SEQ ID NO: 31, SEQ ID NO: 33, SEQ ID NO: 34, SEQ ID NO: 35, SEQ ID NO: 41, SEQ ID NO: 36, SEQ ID NO: 37 in order.

The nucleotide sequence of L1H2-41BB-L6-LiM (from the N-terminus to the C-terminus) is assembled by splicing SEQ ID NO: 48, SEQ ID NO: 30, SEQ ID NO: 31, SEQ ID NO: 33, SEQ ID NO: 34, SEQ ID NO: 35, SEQ ID NO: 41, SEQ ID NO: 36, SEQ ID NO: 37, SEQ ID NO: 45, SEQ ID NO: 30, SEQ ID NO: 38 in order. In addition, the amino acid sequence of BCMA-41BB (from the N-terminus to the C-terminus) is assembled by splicing SEQ ID NO: 3, SEQ ID NO: 6, SEQ ID NO: 7, SEQ ID NO: 9, SEQ ID NO: 10 in order.

The amino acid sequence of BCMA-41BB-L6 (from the N-terminus to the C-terminus) is assembled by splicing SEQ ID NO: 3, SEQ ID NO: 6, SEQ ID NO: 7, SEQ ID NO: 9, SEQ ID NO: 10, SEQ ID NO: 11, SEQ ID NO: 18 in order.

The amino acid sequence of BCMA-41BB-Li (from the N-terminus to the C-terminus) is assembled by splicing SEQ ID NO: 3, SEQ ID NO: 6, SEQ ID NO: 7, SEQ ID NO: 9, SEQ ID NO: 10, SEQ ID NO: 11, SEQ ID NO: 13 in order.

The amino acid sequence of BCMA-41BB-LiM (from the N-terminus to the C-terminus) is assembled by splicing SEQ ID NO: 3, SEQ ID NO: 6, SEQ ID NO: 7, SEQ ID NO: 9, SEQ ID NO: 10, SEQ ID NO: 11, SEQ ID NO: 13, SEQ ID NO: 22, SEQ ID NO: 6, SEQ ID NO: 15 in order.

The amino acid sequence of BCMA-41BB-LiR (from the N-terminus to the C-terminus) is assembled by splicing SEQ ID NO: 3, SEQ ID NO: 6, SEQ ID NO: 7, SEQ ID NO: 9, SEQ ID NO: 10, SEQ ID NO: 11, SEQ ID NO: 16, SEQ ID NO: 17 in order.

The amino acid sequence of BCMA-41BB-L6-Li (from the N-terminus to the C-terminus) is assembled by splicing SEQ ID NO: 3, SEQ ID NO: 6, SEQ ID NO: 7, SEQ ID NO: 9, SEQ ID NO: 10, SEQ ID NO: 11, SEQ ID NO: 18, SEQ ID NO: 12, SEQ ID NO: 13 in order.

The amino acid sequence of BCMA-41BB-L6-LiM (from the N-terminus to the C-terminus) is assembled by splicing SEQ ID NO: 3, SEQ ID NO: 6, SEQ ID NO: 7, SEQ ID NO: 9, SEQ ID NO: 10, SEQ ID NO: 11, SEQ ID NO: 18, SEQ ID NO: 12, SEQ ID NO: 13, SEQ ID NO: 22, SEQ ID NO: 6, SEQ ID NO: 15 in order.

The amino acid sequence of BCMA-41BB-L6-LiR (from the N-terminus to the C-terminus) is assembled by splicing SEQ ID NO: 3, SEQ ID NO: 6, SEQ ID NO: 7, SEQ ID NO: 9, SEQ ID NO: 10, SEQ ID NO: 11, SEQ ID NO: 18, SEQ ID NO: 12, SEQ ID NO: 16, SEQ ID NO: 17 in order.

Correspondingly, the nucleotide sequence of BCMA-41BB (from the N-terminus to the C-terminus) is assembled by splicing SEQ ID NO: 27, SEQ ID NO: 30, SEQ ID NO: 31, SEQ ID NO: 33, SEQ ID NO: 34 in order.

The nucleotide sequence of BCMA-41BB-L6 (from the N-terminus to the C-terminus) is assembled by splicing SEQ ID NO: 27, SEQ ID NO: 30, SEQ ID NO: 31, SEQ ID NO: 33, SEQ ID NO: 34, SEQ ID NO: 35, SEQ ID NO: 41 in order.

The nucleotide sequence of BCMA-41BB-Li (from the N-terminus to the C-terminus) is assembled by splicing SEQ ID NO: 27, SEQ ID NO: 30, SEQ ID NO: 31, SEQ ID NO: 33, SEQ ID NO: 34, SEQ ID NO: 35, SEQ ID NO: 37 in order.

The nucleotide sequence of BCMA-41BB-LiM (from the N-terminus to the C-terminus) is assembled by splicing SEQ ID NO: 27, SEQ ID NO: 30, SEQ ID NO: 31, SEQ ID NO: 33, SEQ ID NO: 34, SEQ ID NO: 35, SEQ ID NO: 37, SEQ ID NO: 45, SEQ ID NO: 30, SEQ ID NO: 38 in order.

The nucleotide sequence of BCMA-41BB-LiR (from the N-terminus to the C-terminus) is assembled by splicing SEQ ID NO: 27, SEQ ID NO: 30, SEQ ID NO: 31, SEQ ID NO: 33, SEQ ID NO: 34, SEQ ID NO: 35, SEQ ID NO: 39, SEQ ID NO: 40 in order.

The nucleotide sequence of BCMA-41BB-L6-Li (from the N-terminus to the C-terminus) is assembled by splicing SEQ ID NO: 27, SEQ ID NO: 30, SEQ ID NO: 31, SEQ ID NO: 33, SEQ ID NO: 34, SEQ ID NO: 35, SEQ ID NO: 41, SEQ ID NO: 36, SEQ ID NO: 37 in order.

The nucleotide sequence of BCMA-41BB-L6-LiM (from the N-terminus to the C-terminus) is assembled by splicing SEQ ID NO: 27, SEQ ID NO: 30, SEQ ID NO: 31, SEQ ID NO: 33, SEQ ID NO: 34, SEQ ID NO: 35, SEQ ID NO: 41, SEQ ID NO: 36, SEQ ID NO: 37, SEQ ID NO: 45, SEQ ID NO: 30, SEQ ID NO: 38 in order.

The nucleotide sequence of BCMA-41BB-L6-LiR (from the N-terminus to the C-terminus) is assembled by splicing SEQ ID NO: 27, SEQ ID NO: 30, SEQ ID NO: 31, SEQ ID NO: 33, SEQ ID NO: 34, SEQ ID NO: 35, SEQ ID NO: 41, SEQ ID NO: 36, SEQ ID NO: 39, SEQ ID NO: 40 in order.

### Example 2. Lentiviral Infection of T Cells

The infection experiment was conducted following a conventional method known to those skilled in the art. The infection steps were briefly described as below:

1. Acquisition of peripheral blood mononuclear lymphocytes (PBMCs), >1×10⁷ cells were obtained through a blood apheresis system.

2. Treatment of cell culture dishes with experimental anti-human CD3/CD28 antibodies.

Anti-human CD3 and anti-human CD28 antibodies were diluted with PBS to a final concentration of 1 ug/ml, the diluted antibody mixture was added into the cell culture dishes to cover the culture dishes, and incubated at room temperature for 2 hours, and then washed once with PBS after 2 hours, ready for use.

3. Activation of T cells

The isolated PBMCs were resuspended with T lymphocyte culture solution (Xvivo15 culture medium+5% FBS+200 U/ml IL2 or Xvivo15 culture medium+5% FBS+20 ng/ml IL21+10 ng/ml IL7) to a final concentration of 1*10⁶ cells/ml, and cultured in the culture dishes treated in step 2 under conditions of 37° C.+5% CO₂ for a time period of 24 hours.

4. Infection of activated T cells

1) Formulation of infection reagents.

A certain amount of T cell culture solution was added into synperonic F108 with a final concentration of 1 mg/ml, mixed uniformly and heated in a water-bath pot to 37° C. for later use.

2) Treatment on culture plates.

1 mg/ml of anti-human CD3 antibody and 0.5 mg/ml of anti-human CD28 antibody were diluted into a suitable amount of PBS buffer at a volume ratio of 1:1000, and retronectin (1 mg/ml) reagent was diluted into the PBS buffer at a volume ratio of 1:40. They were mixed uniformly and then plated into the cell dishes evenly, incubated at room temperature for 2 hours, and washed with PBS after 2 hours for later use.

3) Lentiviral infection of T cells

The activated T cells were diluted with the infection reagents formulated in 1), into which were added lentiviruses following MOI=3, mixed uniformly, and evenly plated in the culture dishes treated in 2).

After infection, the cell density was monitored to maintain the cells at 1*10⁶ cells/ml, which can be amplified by 30-1000 fold generally after 14 days.

### Example 3. Detection of Memory T Cells by Flow Cytometry (GPC3)

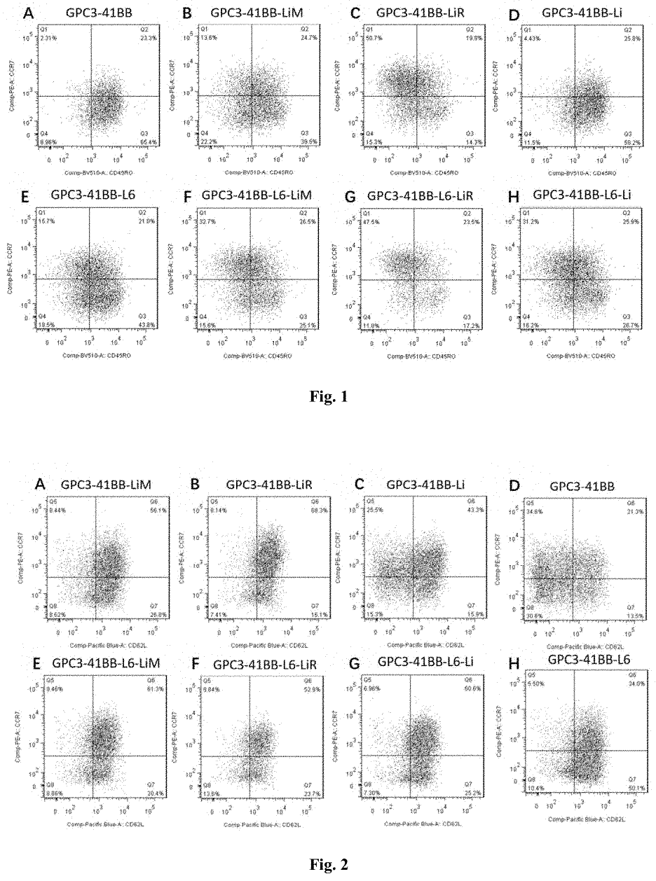

T cells expressing GPC3-41BB, GPC3-41BB-L6, GPC3-41BB-Li, GPC3-41BB-LiM, GPC3-41BB-LiR, GPC3-41BB-L6-Li, GPC3-41BB-L6-LiM, GPC3-41BB-L6-LiR were cultured in vitro, and the expression of memory-related proteins such as CD45RO, CCR7, CD62L in T cells was detected by a BD Canto2 flow cytometer. As shown in FIGS. 1A-1H and FIGS. 2A-2H, the proportions of memory stem cell-like T cells (TSCMs) and central memory T cells (TCMs) (CD45RO-CCR7+; CCR7+CD62L+) in the total T cells expressing GPC3-41BB-L6, GPC3-41BB-Li, GPC3-41BB-LiM, GPC3-41BB-LiR, GPC3-41BB-L6-Li, GPC3-41BB-L6-LiM, GPC3-41BB-L6-LiR were all significantly higher than that in the GPC3-41BB control group, and GPC3-41BB-LiR, GPC3-41BB-L6-Li, GPC3-41BB-L6-LiM, GPC3-41BB-L6-LiR were the most significant.

### Example 4. Detection of Memory T Cells by Flow Cytometry (BCMA)

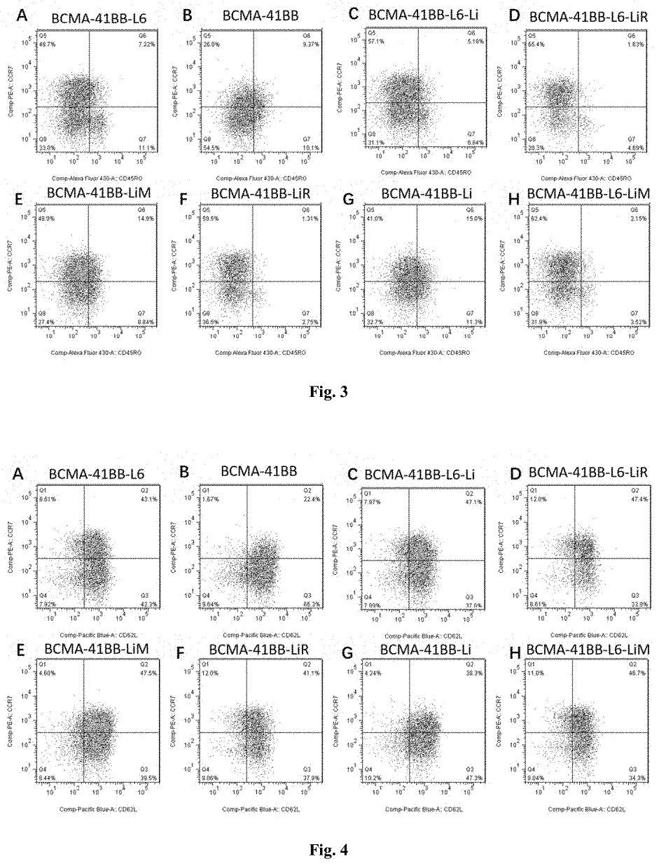

T cells expressing BCMA-41BB, BCMA-41BB-L6, BCMA-41BB-Li, BCMA-41BB-LiM, BCMA-41BB-LiR, BCMA-41BB-L6-Li, BCMA-41BB-L6-LiM, BCMA-41BB-L6-LiR were cultured in vitro, and the expression of memory-related proteins such as CD45RO, CCR7, CD62L in T cells was detected by a BD Canto2 flow cytometer. As shown in FIGS. 3A-3H and FIGS. 4A-4H, the proportions of memory stem cell-like T cells (TSCMs) and central memory T cell (TCMs) (CD45RO-CCR7+; CCR7+CD62L+) in the total T cells expressing BCMA-41BB-L6, BCMA-41BB-Li, BCMA-41BB-LiM, BCMA-41BB-LiR, BCMA-41BB-L6-Li, BCMA-41BB-L6-LiM, BCMA-41BB-L6-LiR were all significantly higher than that in the BCMA-41BB control group.

### Example 5. Detection of Memory T Cells in CD4 Cells by Flow Cytometry

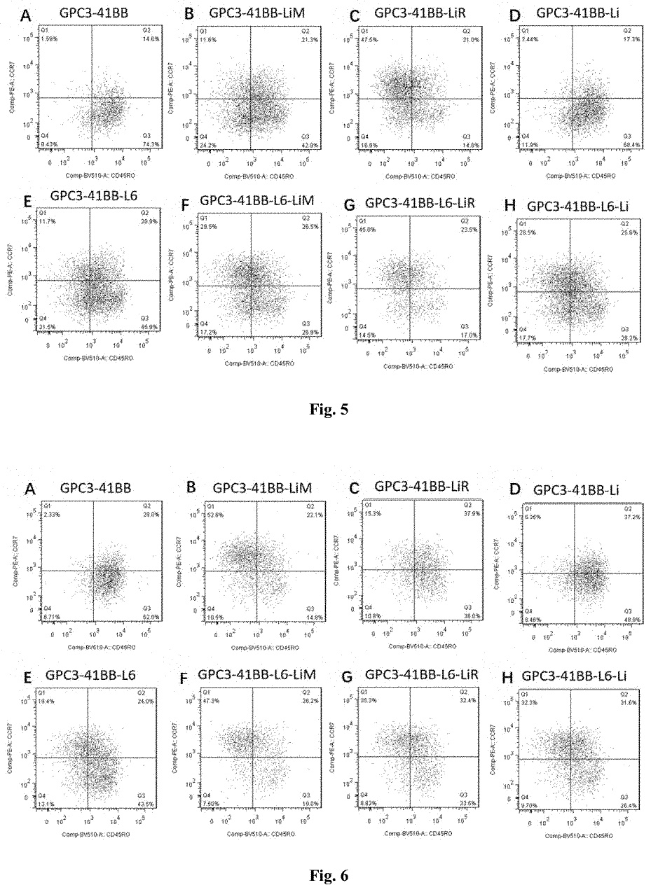

T cells expressing GPC3-41BB, GPC3-41BB-L6, GPC3-41BB-Li, GPC3-41BB-LiM, GPC3-41BB-LiR, GPC3-41BB-L6-Li, GPC3-41BB-L6-LiM, GPC3-41BB-L6-LiR were cultured in vitro, and the expression of CD45RO protein in T cells was detected by a BD Canto2 flow cytometer. As shown in FIGS. 5A-5H, the proportions of memory stem cell-like T cells (TSCMs) (CD45RO-CCR7+) in the total CD4 T cells expressing GPC3-41BB-L6, GPC3-41BB-Li, GPC3-41BB-LiM, GPC3-41BB-LiR, GPC3-41BB-L6-Li, GPC3-41BB-L6-LiM, GPC3-41BB-L6-LiR were all significantly higher than that in the GPC3-41BB control group.

### Example 6. Detection of Memory T Cells in CD8 Cells by Flow Cytometry

T cells expressing GPC3-41BB, GPC3-41BB-L6, GPC3-41BB-Li, GPC3-41BB-LiM, GPC3-41BB-LiR, GPC3-41BB-L6-Li, GPC3-41BB-L6-LiM, GPC3-41BB-L6-LiR were cultured in vitro, and the expression of CD3, CD4, CD8, CD45RO, CD45RA, CD62L, CCR7, CD95, CD122, CD127, CD27, CD28 proteins in T cells was detected by a BD Canto2 flow cytometer. As shown in FIGS. 6A-6H, the proportions of memory stem cell-like T cells (TSCMs) (CD45RO-CCR7+) in the total CD8 T cells expressing GPC3-41BB-L6, GPC3-41BB-Li, GPC3-41BB-LiM, GPC3-41BB-LiR, GPC3-41BB-L6-Li, GPC3-41BB-L6-LiM, GPC3-41BB-L6-LiR were all significantly higher than that in the GPC3-41BB control group.

### Example 7: Detection on the Expression of Negative Regulatory Proteins in T Cells by Flow Cytometry

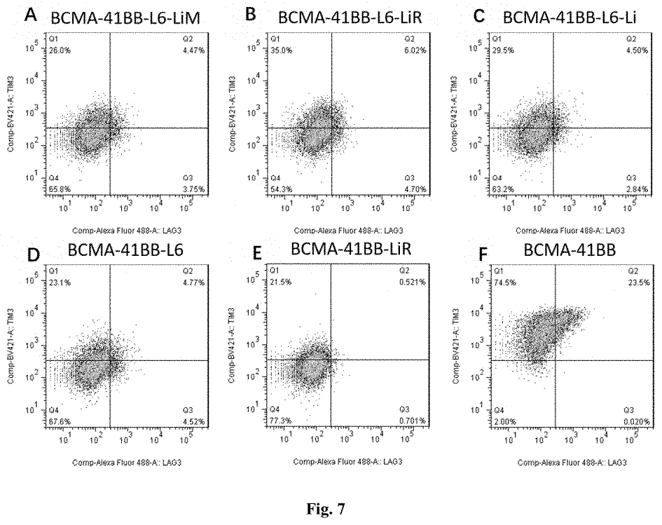

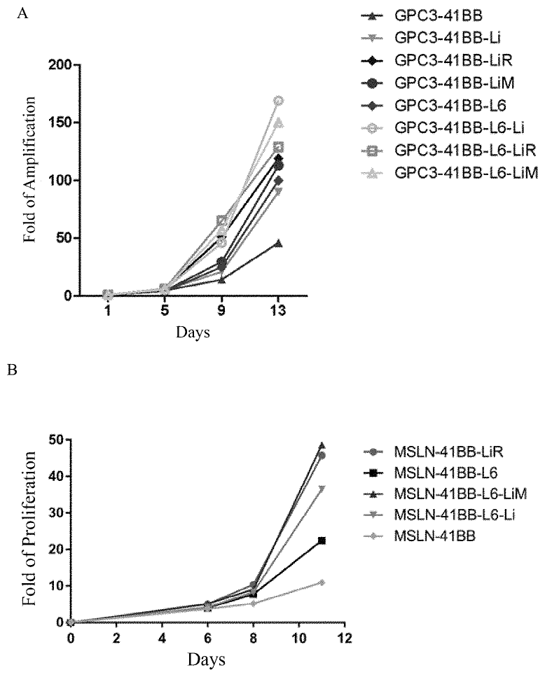

T cells expressing BCMA-41BB, BCMA-41BB-L6, BCMA-41BB-Li, BCMA-41BB-LiM, BCMA-41BB-LiR, BCMA-41BB-L6-Li, BCMA-41BB-L6-LiM, BCMA-41BB-L6-LiR were cultured in vitro, and the expression of CD3, CD4, CD8, PD1, PDL1, TIM3, LAG3 proteins in T cells was detected by a BD Canto2 flow cytometer. As shown in FIGS. 7A-7F, the expression of negative regulatory proteins in T cells expressing BCMA-41BB-L6, BCMA-41BB-Li, BCMA-41BB-LiM, BCMA-41BB-LiR, BCMA-41BB-L6-Li, BCMA-41BB-L6-LiM, BCMA-41BB-L6-LiR was significantly lower than that in the BCMA-41BB control group.

### Example 8: Detection of T Cell Amplification

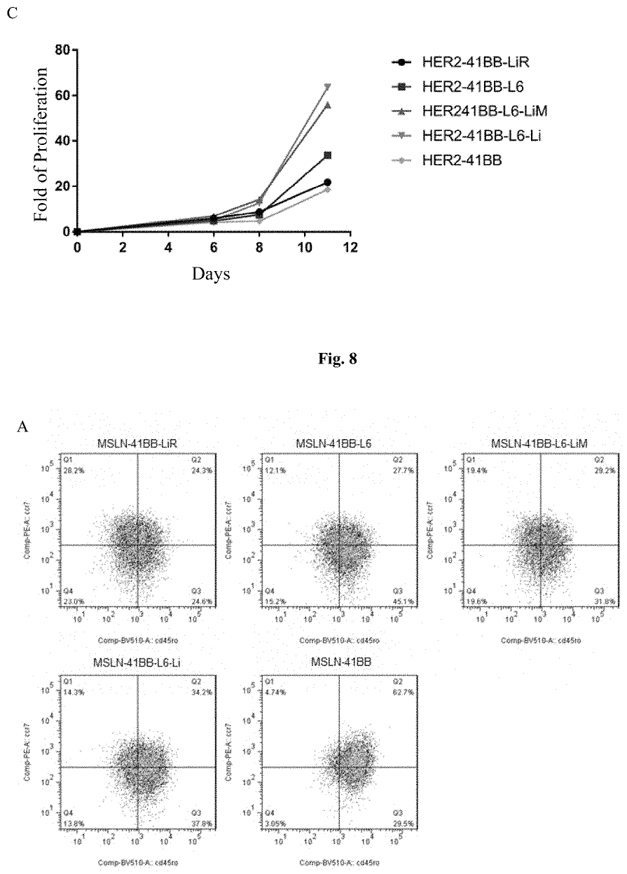

T cells expressing GPC3-41BB, GPC3-41BB-L6, GPC3-41BB-Li, GPC3-41BB-LiM, GPC3-41BB-LiR, GPC3-41BB-L6-Li, GPC3-41BB-L6-LiM, GPC3-41BB-L6-LiR, MSLN-41BB, MSLN-41BB-L6, MSLN-41BB-LiR, MSLN-41BB-L6-Li, MSLN-41BB-L6-LiM, HER2-41BB, HER2-41BB-L6, HER2-41BB-LiR, HER2-41BB-L6-Li, HER2-41BB-L6-LiM were cultured in vitro, stained with trypan blue (GIBICO) and then counted by a Cellometer Mini automatic cell counter to record the amplification capacity of the cells. As shown in FIGS. 8A-8C, the amplification capacities of T cells expressing GPC3-41BB-L6, GPC3-41BB-Li, GPC3-41BB-LiM, GPC3-41BB-LiR, GPC3-41BB-L6-Li, GPC3-41BB-L6-LiM, GPC3-41BB-L6-LiR, MSLN-41BB-L6, MSLN-41BB-LiR, MSLN-41BB-L6-Li, MSLN-41BB-L6-LiM, HER2-41BB-L6, HER2-41BB-LiR, HER2-41BB-L6-Li, HER2-41BB-L6-LiM were all significantly higher than that in the GPC3-41BB, MSLN-41BB and HER2-41BB control groups.

### Example 9. Detection of Memory T Cells by Flow Cytometry (MSLN)

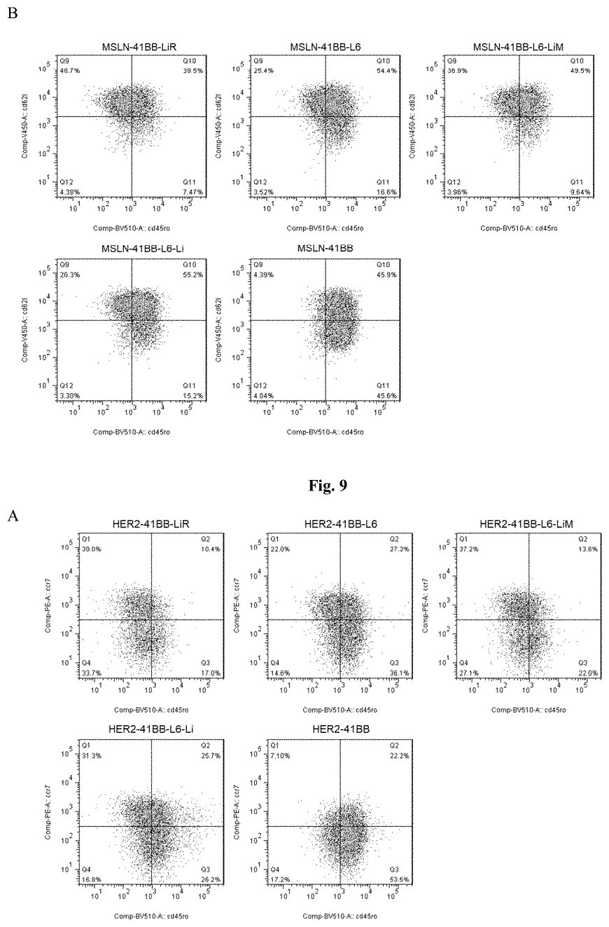

T cells expressing MSLN-41BB, MSLN-41BB-L6, MSLN-41BB-LiR, MSLN-41BB-L6-Li, MSLN-41BB-L6-LiM were cultured in vitro, and the expression of memory-related proteins such as CD45RO, CCR7, CD62L in T cells was detected by flow cytometry. As shown in FIG. 9A and FIG. 9B, the proportions of memory stem cell-like T cells (TSCMs) (CD45RO-CCR7+; CD45RO-CD62L+) in the total T cells expressing MSLN-41BB-L6, MSLN-41BB-LiR, MSLN-41BB-L6-Li, MSLN-41BB-L6-LiM were all significantly higher than that in the MSLN-41BB control group, especially those in the MSLN-41BB-LiR and MSLN-41BB-L6-LiM groups.

### Example 10. Detection of Memory T Cells by Flow Cytometry (HER2)

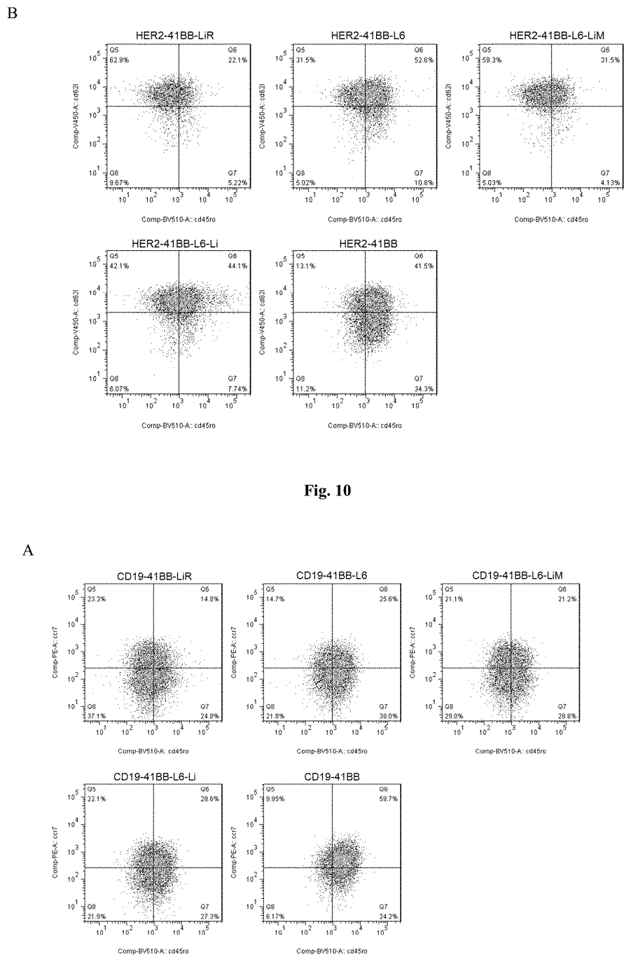

T cells expressing HER2-41BB, HER2-41BB-L6, HER2-41BB-LiR, HER2-41BB-L6-Li, HER2-41BB-L6-LiM were cultured in vitro, and the expression of memory-related proteins such as CD45RO, CCR7, CD62L in T cells was detected by flow cytometry. As shown in FIG. 10A and FIG. 10B, the proportions of memory stem cell-like T cells (TSCMs) (CD45RO-CCR7+; CD45RO-CD62L+) in the total T cells expressing HER2-41BB-L6, HER2-41BB-LiR, HER2-41BB-L6-Li, HER2-41BB-L6-LiM were all significantly higher than that in the HER2-41BB control group, especially those in the HER2-41BB-LiR, HER2-41BB-L6-Li and HER2-41BB-L6-LiM groups.

### Example 11. Detection of Memory T Cells by Flow Cytometry (CD19)

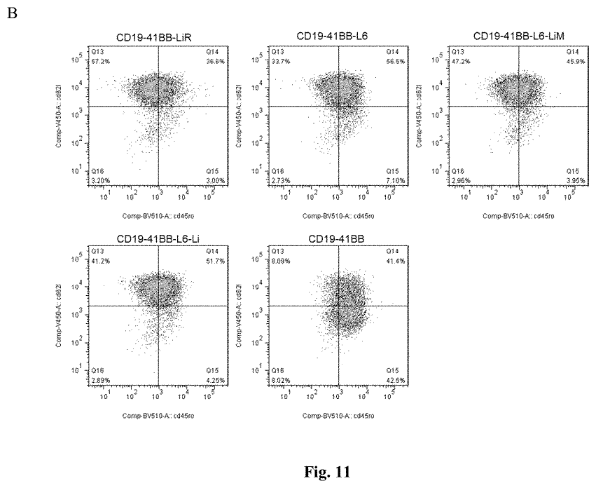

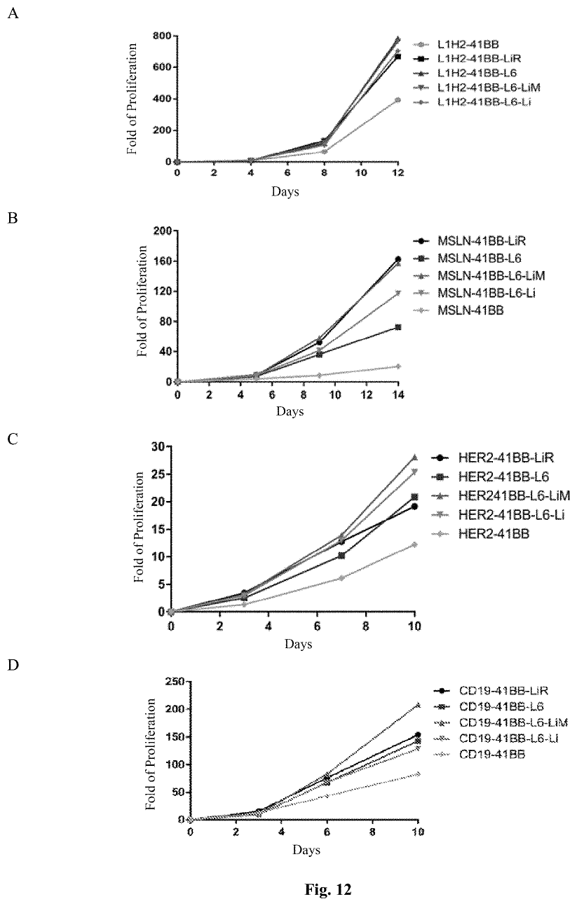

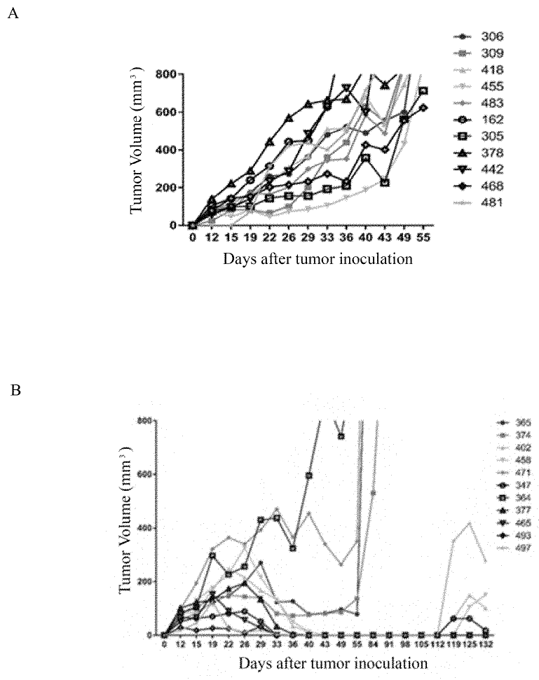

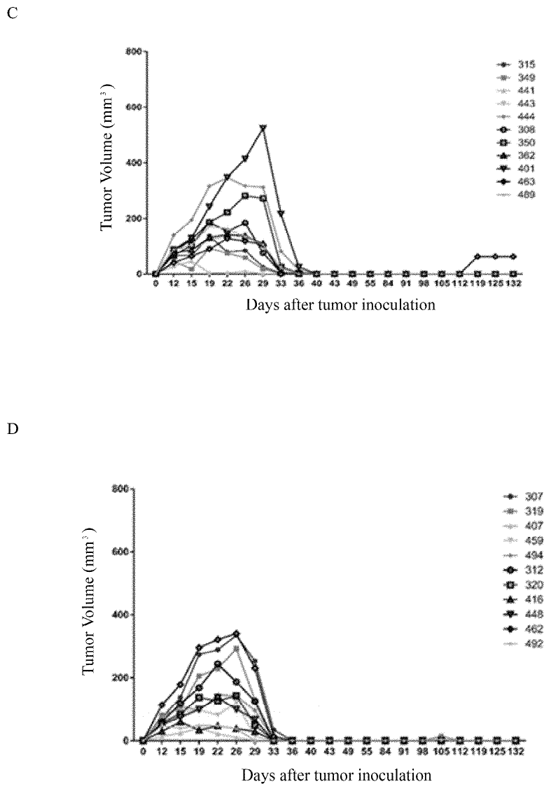

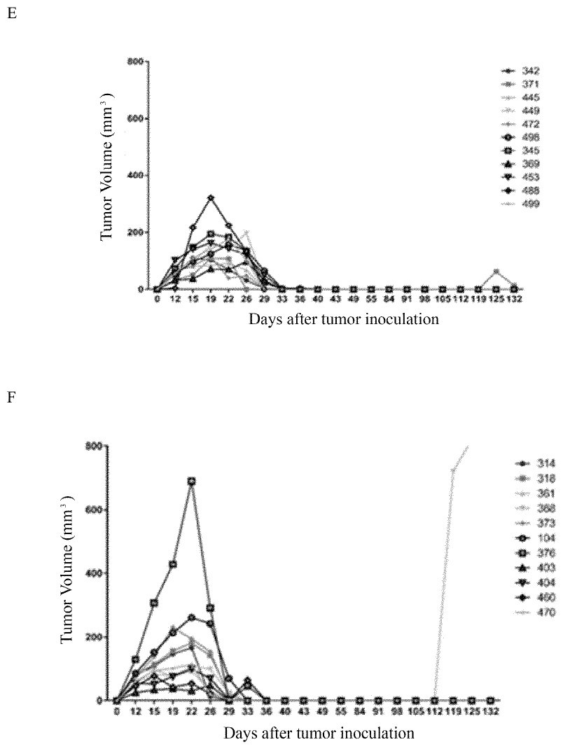

T cells expressing CD19-41BB, CD19-41BB-L6, CD19-41BB-LiR, CD19-41BB-L6-Li, CD19-41BB-L6-LiM were cultured in vitro, and the expression of memory-related proteins such as CD45RO, CCR7, CD62L in T cells was detected by flow cytometry. As shown in FIG. 11A and FIG. 11B, the proportions of memory stem cell-like T cells (TSCMs) (CD45RO-CCR7+; CD45RO-CD62L+) in the total T cells expressing CD19-41BB-L6, CD19-41BB-LiR, CD19-41BB-L6-Li, CD19-41BB-L6-LiM were all significantly higher than that in the CD19-41BB control group, especially those in the CD19-41BB-LiR, CD19-41BB-L6-Li and CD19-41BB-L6-LiM groups.

### Example 12. Detection by Repeated Stimulation (MSLN, L1H2 [GPC3-Targeted], HER2, CD19)

T cells expressing L1H2-41BB, L1H2-41BB-L6, L1H2-41BB-LiR, L1H2-41BB-L6-Li, L1H2-41BB-L6-LiM, MSLN-41BB, MSLN-41BB-L6, MSLN-41BB-LiR, MSLN-41BB-L6-Li, MSLN-41BB-L6-LiM, HER2-41BB, HER2-41BB-L6, HER2-41BB-LiR, HER2-41BB-L6-Li, HER2-41BB-L6-LiM, CD19-41BB, CD19-41BB-L6, CD19-41BB-LiR, CD19-41BB-L6-Li, CD19-41BB-L6-LiM were cultured in vitro, for which the positive rates were detected on day 9, and then the positive rates were adjusted with uninfected T cells to consistent. 3×10e5 CAR-positive cells and 3×10e5 irradiated target cells were taken for the first round of co-incubation stimulation (the corresponding target cells of L1H2 CAR-T are Huh7, the corresponding target cells of MSLN CAR-T are OVCAR3, the corresponding target cells of HER2 CAR-T are SKOV3, and the corresponding target cells of CD19 CAR-T are Raji), and counted every 3-5 days. And 3×10e5 total cells and 3×10e5 irradiated target cells were taken for the second round of co-incubation stimulation, the above operation was repeated after 3-5 days for the third round of co-incubation stimulation, and the above operation was repeated after 3-5 days for the fourth round of co-incubation stimulation. The counting results were converted to form a repeated stimulation amplification curve. As shown from the results in FIGS. 12A-12D, the capacities of amplification by repeated stimulation in the L6, LiR, L6-Li, L6-LiM structural groups were all significantly higher than that in the 41BB control group, indicating better amplification capacity and tumor treatment ability in vivo.

### Example 13. CAR-T Tumor Suppression and Elimination Experiment (GPC3-Targeted)

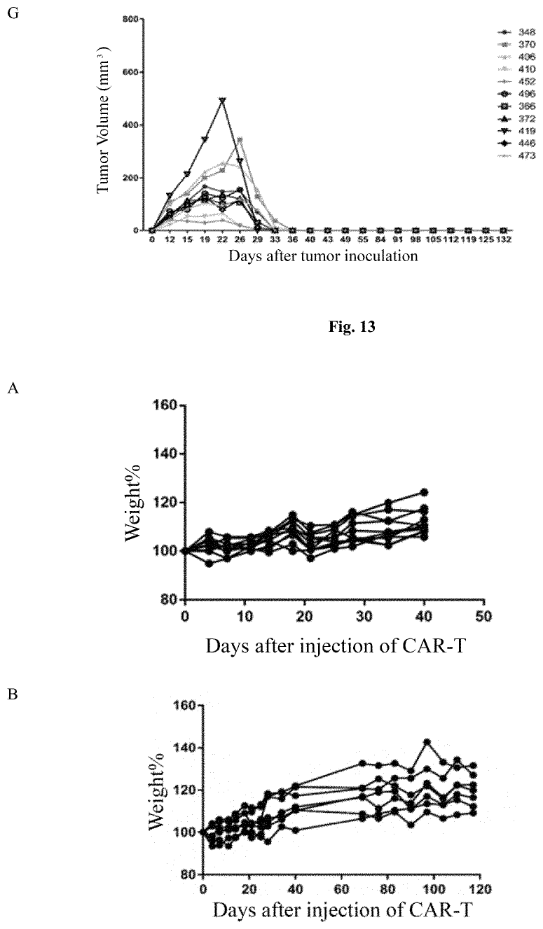

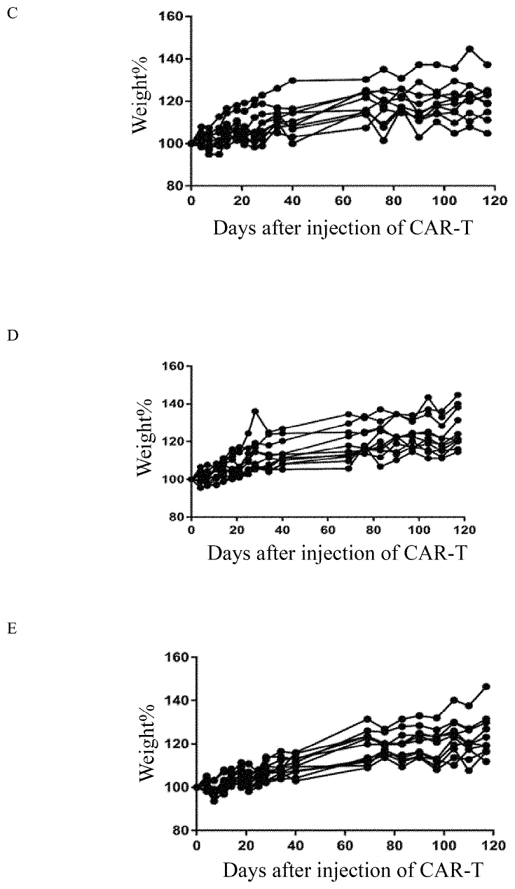

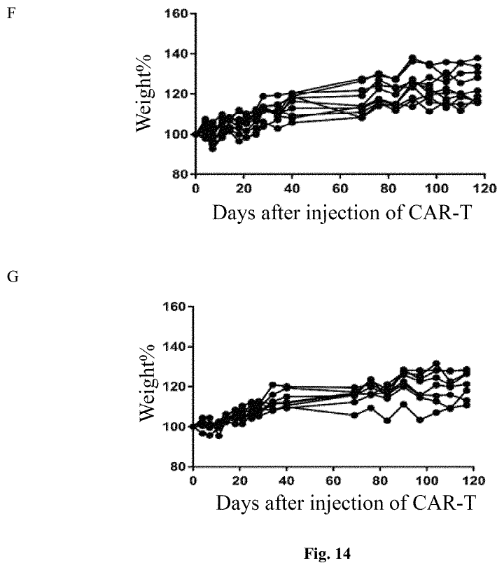

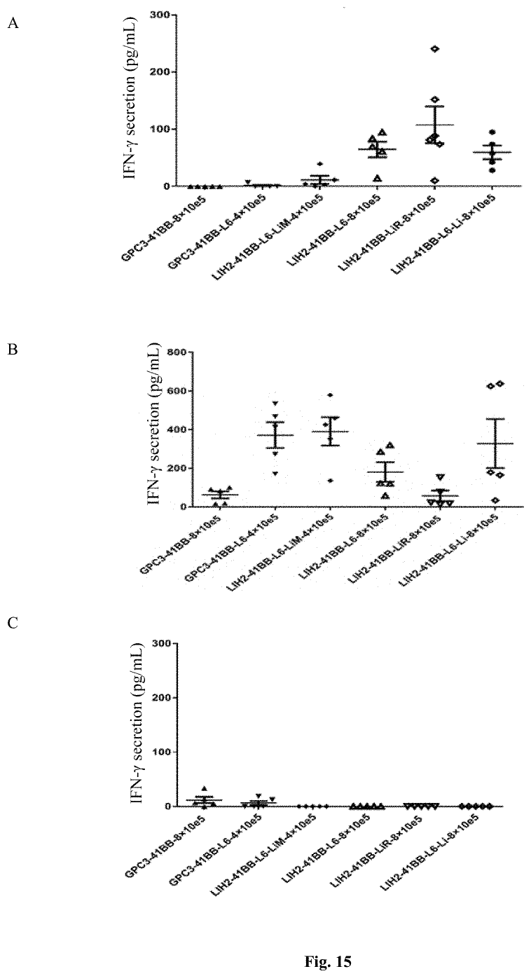

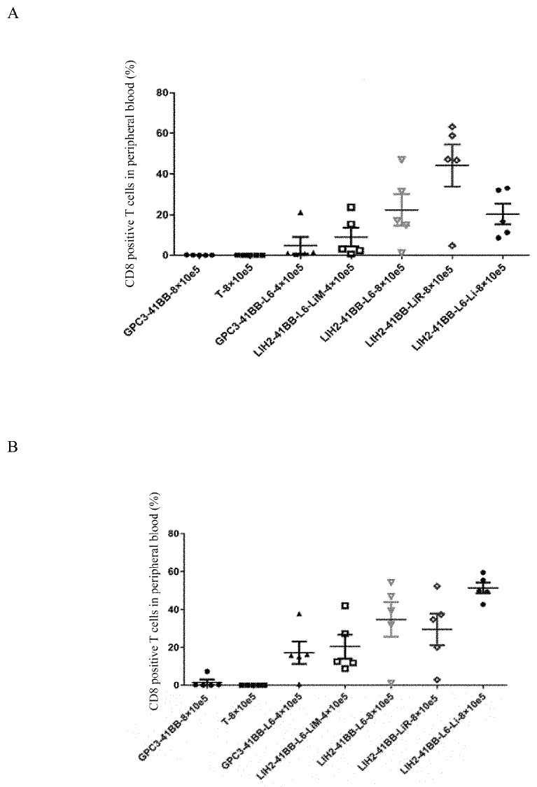

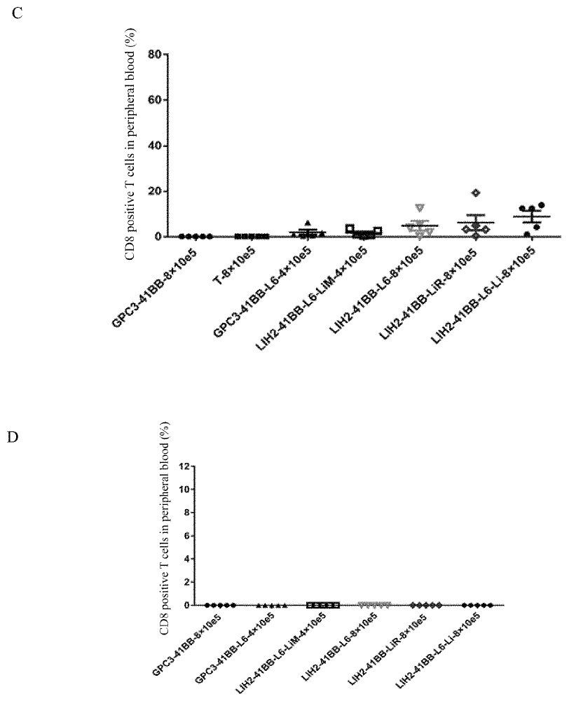

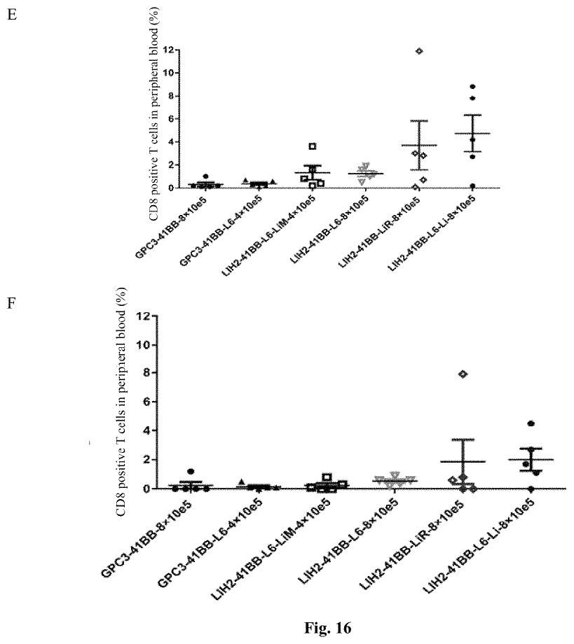

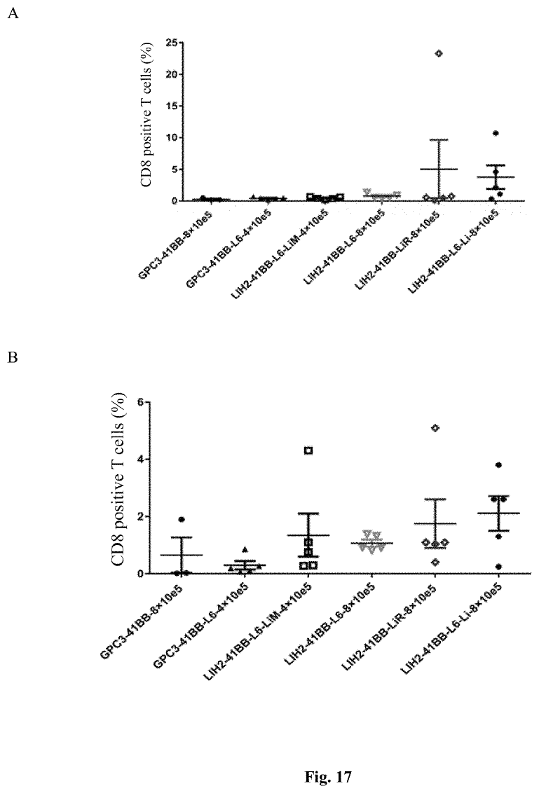

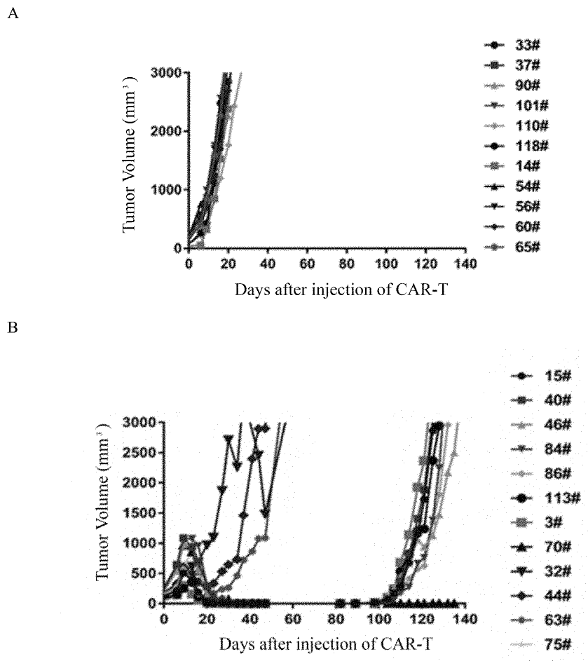

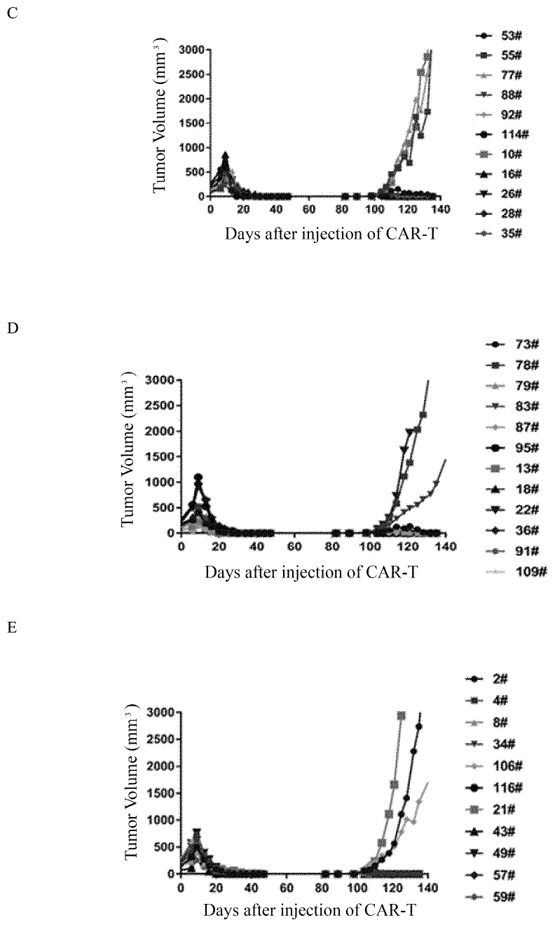

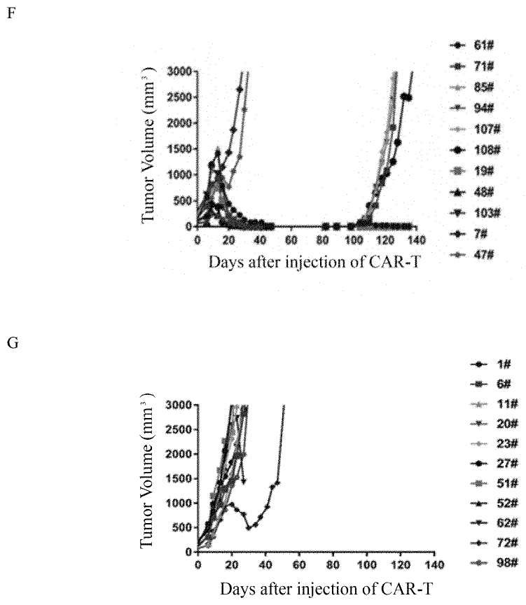

T cells expressing GPC3-41BB, GPC3-41BB-L6, L1H2-41BB-L6, L1H2-41BB-LiR, L1H2-41BB-L6-Li, L1H2-41BB-L6-LiM and T cells not infected with CAR-T were subjected to a single tail vein injection of CAR-T into 6-week-old NSG mice with subcutaneous tumor-bearing of Huh7 tumors (1×10e7/mouse) to 80-150 mm³ size at doses of 4×10e5 and 8×10e5 CAR-positive cells, with 11 mice in each group. The body weight (FIGS. 14A-14G show the body weight changes of mice that have been injected with 8×10e5 T cells uninfected with CAR-T, T cells infected with 8×10e5 GPC3-41BB, T cells infected with 4×10e5 GPC3-41BB-L6, T cells infected with 4×10e5 L1H2-41BB-L6-LiM, T cells infected with 8×10e5 L1H2-41BB-L6, T cells infected with 8×10e5 L1H2-41BB-LiR, T cells infected with 8×10e5 L1H2-41BB-L6-Li, respectively) and the tumor sizes (FIGS. 13A-13G show the tumor sizes of mice that have been injected with T cells not infected with 8×10e5 CAR-T, T cells infected with 8×10e5 GPC3-41BB, T cells infected with 4×10e5 GPC3-41BB-L6, T cells infected with 4×10e5 L1H2-41BB-L6-LiM, T cells infected with 8×10e5 L1H2-41BB-L6, T cells infected with 8×10e5 L1H2-41BB-LiR, T cells infected with 8×10e5 L1H2-41BB-L6-Li, respectively) were measured twice a week. On days 4, 11, 17 after the injection of CAR-T, five mice were randomly selected from each group to take anticoagulated blood (heparin sodium anticoagulation) from the tail vein for the detection of IFNg in blood (BD human th1/th2 CBA kit) (FIGS. 15A-15C show the results on days 4, 11, 17 in order). On days 11, 17, 26 after the injection of CAR-T, five mice were randomly selected from each group to take anticoagulated blood (heparin sodium anticoagulation) from the tail vein for the detection of human CD8 cell content in blood by flow cytometry (FIGS. 16A-16C show the results on days 11, 17, 26 in order). On day 85 after the injection of CAR-T (day 98 after the injection of tumor), the other side of the back of the mice without recurrence after tumor elimination was subjected to a secondary tumor-bearing of Huh7 (1×10e7/mouse) to simulate tumor recurrence, and the tumor formation was observed (FIG. 13). On days 91, 98, 105, five mice were randomly selected from each group of secondary tumor-bearing mice to take anticoagulated blood (heparin sodium anticoagulation) from the tail vein for the detection of human CD8 cell content in blood by flow cytometry (FIGS. 16D-16F show the results on days 91, 98, 105 in order). On day 132, five mice were randomly selected from each group of secondary tumor-bearing mice to take spleen and bone marrow for the detection of human CD8 cell content by flow cytometry (FIGS. 17A-17B), the experiment is over. The experimental results show that, compared with the GC33-41BB control group, the groups of GPC3-41BB-L6, L1H2-41BB-L6, L1H2-41BB-LiR, L1H2-41BB-L6-Li, L1H2-41BB-L6-LiM all show more significant tumor suppression and elimination effects and anti-recurrence ability (FIG. 13), while none of them show any obvious toxic side effects (FIG. 14, no obvious weight loss); the results of in vivo cytokine assay show that the groups of GPC3-41BB-L6, L1H2-41BB-L6, L1H2-41BB-LiR, L1H2-41BB-L6-Li, L1H2-41BB-L6-LiM all have higher factor secretion, which is beneficial to promote the in vivo amplification of CAR-T, wherein L1H2-41BB-LiR and L1H2-41BB-L6-Li are the most significant (FIGS. 15A-15C); the results of in vivo cell proliferation assay show that the groups of GPC3-41BB-L6, L1H2-41BB-L6, L1H2-41BB-LiR, L1H2-41BB-L6-Li, L1H2-41BB-L6-LiM all have higher cell proliferation capability, wherein L1H2-41BB-LiR and L1H2-41BB-L6-Li are the most significant (FIGS. 16A-16C). In addition, after secondary tumor-bearing, the levels of in vivo CAR-T cells that have restored to the baseline value in the groups of L1H2-41BB-L6, L1H2-41BB-LiR, L1H2-41BB-L6-Li, L1H2-41BB-L6-LiM have obvious secondary proliferation, which is positively correlated with its better anti-recurrence ability, wherein L1H2-41BB-LiR and L1H2-41BB-L6-Li are the most significant (FIGS. 16D-16F). Meanwhile, the spleen and bone marrow assay after secondary tumor-bearing show that the groups of L1H2-41BB-L6, L1H2-41BB-LiR, L1H2-41BB-L6-Li, L1H2-41BB-L6-LiM have more memory T cells homing to lymph nodes, wherein L1H2-41BB-L6-Li is the most significant.

### Example 14. CAR-T Tumor Suppression and Elimination Experiment (BCMA-Targeted)

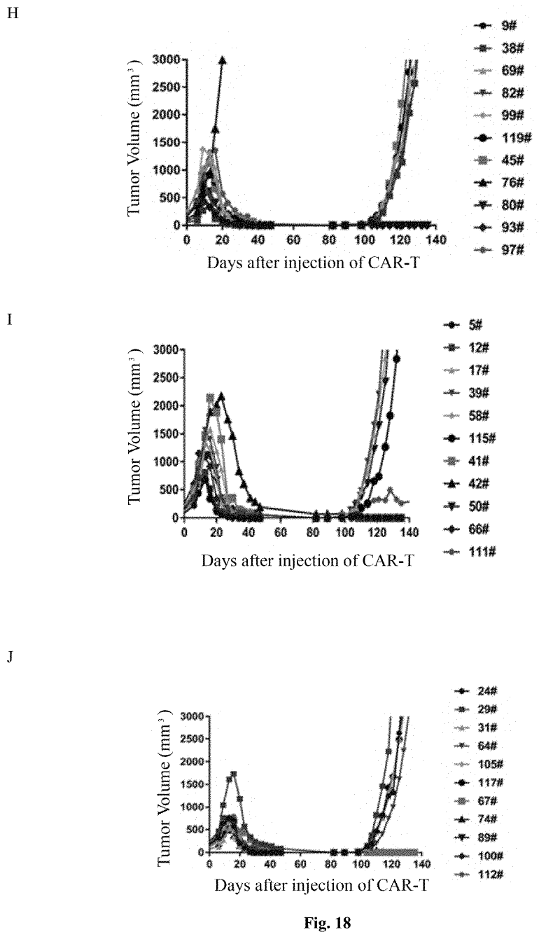

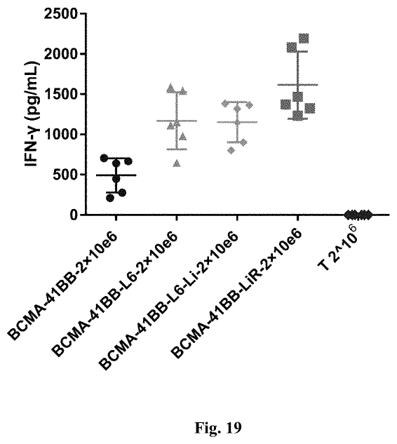

T cells expressing BCMA-41BB, BCMA-41BB-L6, BCMA-41BB-LiR, BCMA-41BB-L6-Li, BCMA-41BB-L6-LiM and T cells not infected with CAR-T were subjected to a single tail vein injection of CAR-T into 6-week-old NSG mice with subcutaneous tumor-bearing of MM1.S tumors (1×10e7/mouse) to 80-150 mm³ size at doses of 6×10e5 and 2×10e6 CAR-positive cells, with 11 mice in each group. The tumor size was measured twice a week (FIGS. 18A-18J). On day 8 after the injection of CAR-T, six mice were randomly selected from each 2×10e6 dose group to take anticoagulated blood (heparin sodium anticoagulation) from the tail vein for the detection of IFNg in blood (BD human th1/th2 CBA kit) (FIG. 19). On day 80 after the injection of CAR-T, the other side of the back of the mice without recurrence after tumor elimination was subjected to a secondary tumor-bearing of MM1.S (1×10e7/mouse) to simulate tumor recurrence, and the tumor formation was observed (FIGS. 18B-18J). The results of the control group inoculated with T cells not infected with CAR-T are shown in FIG. 18A. The experimental results show that, compared with the control group inoculated with 2×10e6 BCMA-41BB positive cells (FIG. 18B) and the control group inoculated with 6×10e5 BCMA-41BB positive cells (FIG. 18 G), the experimental groups inoculated with 2×10e6 BCMA-41BB-L6 positive cells (FIG. 18C) and inoculated with 6×10e5 BCMA-41BB-L6 positive cells (FIG. 18H), the experimental groups inoculated with 2×10e6 BCMA-41BB-LiR positive cells (FIG. 18E) and inoculated with 6×10e5 BCMA-41BB-LiR positive cells (FIG. 18J), the experimental groups inoculated with 2×10e6 BCMA-41BB-L6-Li positive cells (FIG. 18D) and inoculated with 6×10e5 BCMA-41BB-L6-Li positive cells (FIG. 18I), and the experimental group inoculated with 6×10e5 BCMA-41BB-L6-LiM positive cells (FIG. 18F) all show more significant tumor suppression and elimination effects and anti-recurrence ability; the results of in vivo cytokine assay show that compared with BCMA-41BB group, the groups of BCMA-41BB-L6, BCMA-41BB-LiR, BCMA-41BB-L6-Li all have higher factor secretion, which is beneficial to promote the in vivo amplification of CAR-T, wherein the BCMA-41BB-LiR group is the most significant (FIG. 19). In addition, after secondary tumor-bearing, significant recurrence occurred in all of the groups due to the high tumor-bearing doses, but the numbers of recurrence in various dose groups of BCMA-41BB-L6, BCMA-41BB-LiR, BCMA-41BB-L6-Li, BCMA-41BB-L6-LiM were all significantly lower than that in the high dose group of BCMA-41BB; in low dose groups, BCMA-41BB-L6-Li, BCMA-41BB-L6-LiM are slightly better than BCMA-41BB-LiR, and significantly better than BCMA-41BB-L6, while a low dose of BCMA-41BB cannot suppress tumors at all.

The foregoing detailed description is provided by way of explanation and examples, and is not intended to limit the scope of the appended claims. Various changes of the embodiments currently enumerated in this application will be apparent to those of ordinary skills in the art, and are reserved within the scope of the appended claims and their equivalents.

1. A modified immune cell, comprising a chimeric antigen receptor and/or a coding element therefor, or comprising a T cell receptor and/or a coding element therefor, and the modified immune cell further comprises:
a leptin and/or a functional fragment thereof; and/or,
a leptin receptor and/or a functional fragment thereof, and the expression quantity of the leptin receptor and/or the functional fragment thereof in the modified immune cell is increased compared with that in an immune cell without the corresponding modification.

2-3. (canceled)

4. The modified immune cell according to claim 1, wherein the leptin and/or the functional fragment thereof, and/or
a leptin receptor and/or a functional fragment thereof is derived from a human.

5. (canceled)

6. The modified immune cell according to claim 1, wherein the leptin is a secretory leptin or a membrane-bound leptin.

7. The modified immune cell according to claim 1, wherein the leptin and/or the functional fragment thereof comprises an amino acid sequence as set forth in any one of SEQ ID NOs: 13-14.

8. The modified immune cell according to claim 1, wherein the functional fragment of the leptin receptor comprises a domain of the leptin receptor selected from the group consisting of: an intracellular region, a BOX 1 region, an FN3 region, a C2 region, and a transmembrane region.

9. The modified immune cell according to claim 1, wherein the leptin receptor and/or the functional fragment thereof comprises an amino acid sequence as set forth in any one of SEQ ID NOs: 15-17.

10.-23. (canceled)

24. A pharmaceutical composition, comprising the modified immune cell according to claim 1, and optionally a pharmaceutically acceptable carrier.

25-26. (canceled)

27. A method for promoting immune cell proliferation, or promoting the production of memory immune cells, or enhancing the killing ability of an immune cell against a tumor, the method comprising:
1) providing a modified immune cell comprising a chimeric antigen receptor and/or a coding element therefor, or comprising a T cell receptor and/or a coding element therefor; and
2) allowing the modified immune cell of 1) to express leptin and/or a functional fragment thereof, and/or increasing the expression quantity of a leptin receptor and/or a functional fragment thereof in the modified immune cell of 1).

28-53. (canceled)

54. A method for inhibiting tumor cell proliferation and/or killing tumor cell, comprising: contacting the modified immune cell of claim 1 with the tumor cell.

55-56. (canceled)

57. The method according to claim 54, wherein the tumor comprises at least one selected from the group consisting of B lymphocytic leukemia, breast cancer, stomach cancer, pancreatic cancer, multiple myeloma, mesothelioma, lung cancer, and liver cancer.

58. A modified immune cell, comprising leptin and/or a functional fragment thereof, and/or, a leptin receptor and/or a functional fragment thereof, and a low-density lipoprotein-receptor-related protein or a fragment thereof.

59-62. (canceled)

63. The modified immune cell according to claim 58, wherein the leptin and/or the functional fragment thereof comprises an amino acid sequence as set forth in any one of SEQ ID NOs: 13-14.

64. The modified immune cell according to claim 58, wherein the functional fragment of the leptin receptor comprises a domain of the leptin receptor selected from the group consisting of: an intracellular region, a BOX 1 region, an FN3 region, a C2 region, and a transmembrane region.

65. The modified immune cell according to claim 58, wherein the leptin receptor and/or the functional fragment thereof comprises an amino acid sequence as set forth in any one of SEQ ID NOs: 15-17.

66. The modified immune cell according to claim 58, wherein the low-density lipoprotein-receptor-related protein or the fragment thereof is selected from the group consisting of: a low-density lipoprotein-receptor-related protein 6 or a truncated body thereof, and a low-density lipoprotein-receptor-related protein 5 or a truncated body thereof.

67. The modified immune cell according to claim 66, wherein the truncated body of the low-density lipoprotein-receptor-related protein 6 comprises a functional domain selected from the group consisting of: an intracellular region of the low-density lipoprotein-receptor-related protein 6 and a transmembrane region of the low-density lipoprotein-receptor-related protein 6; and/or,
wherein the truncated body of the low-density lipoprotein-receptor-related protein 5 comprises a functional domain selected from a group consisting of: an intracellular region of the low-density lipoprotein-receptor-related protein 5 and a transmembrane region of the low-density lipoprotein-receptor-related protein 5

68. (canceled)

69. The modified immune cell according to claim 58, wherein the low-density lipoprotein-receptor-related protein or the fragment thereof comprises an amino acid sequence as set forth in any one of SEQ ID NOs: 18-21.

70. The modified immune cell according to claim 58, further comprising a chimeric antigen receptor or a T cell receptor.

71-85. (canceled)

86. A pharmaceutical composition, comprising the modified immune cell according to claim 58 and optionally a pharmaceutically acceptable carrier.

87. (canceled)

88. A method for promoting immune cell proliferation, or promoting the production of memory immune cells, or enhancing the killing ability of an immune cell against a tumor, the method comprising:
allowing a modified immune cell to express leptin and/or a functional fragment thereof, and/or increasing the expression quantity of a leptin receptor and/or a functional fragment thereof in the modified immune cell, and
allowing the modified immune cell to express a low-density lipoprotein-receptor-related protein or a fragment thereof.

89-122. (canceled)

123. A method for inhibiting tumor cell proliferation and/or killing a tumor cell, comprising: contacting the modified immune cell according to claim 58 with the tumor cell.

124-125. (canceled)

126. The method according to claim 123, wherein the tumor comprises at least one selected from the group consisting of B lymphocytic leukemia, breast cancer, stomach cancer, pancreatic cancer, multiple myeloma, mesothelioma, lung cancer, and liver cancer.
